(19) United States
(12) Patent Application Publication
(10) Pub. No.: US 2019/0365799 A1
(43) Pub. Date: Dec. 5, 2019

(54) THERAPIES FOR THE TREATMENT OF LIDOCAINE-INEFFECTIVE AND HYPOKALEMIC CONDITIONS

(71) Applicant: AlkaliDx, Inc., Chestnut Hill, MA (US)

(72) Inventor: Michael M. SEGAL, Chestnut Hill, MA (US)

(21) Appl. No.: 16/349,824

(22) PCT Filed: Nov. 22, 2017
(86) PCT No.: PCT/US17/63009

§ 371 Date: May 14, 2019

Related U.S. Application Data
(60) Provisional application No. 62/425,512, filed on Nov. 22, 2016.
(60) Provisional application No. 62/449,799, filed on Jan. 24, 2017.

(51) Int. Cl. A61K 33/14; A61K 45/06; A61K 31/554; A23L 33/16

(52) U.S. Cl. CPC A61K 33/14; A23L 33/16; A61K 31/554; A61K 45/06

(57) ABSTRACT

The invention provides methods of treating a human patient having a Lidocaine-lneffective Condition or a hypokalemic condition by administration of either a potassium-elevating agent or potassium, alone or in combination with additional agents. Patients amenable to the treatment regimens described herein include those having a diagnosis of a hypokalemic condition, disorder of attention Asperger Syndrome, Sensory Overstimulation Syndrome (SOS), Sensory Processing Disorder, Sensory Integration Disorder, Fibromyalgia, various pain syndromes and/or Premenstrual Syndrome. The invention additionally features pharmaceutical compositions and kits for the treatment of such conditions.

### BACKGROUND OF THE INVENTION

Disorders of attention, Asperger Syndrome, pain syndromes, and Premenstrual Syndrome are conditions that have traditionally been considered as separate pathologies.

Many patients having these disorders are not able to compensate adequately; they often develop low self-esteem and must receive appropriate treatment to achieve their full intellectual and social potential. Attention deficit, with or without hyperactivity, can result in poor performance in school and jobs. If left untreated, these conditions may exact a significant hardship on affected individuals, loved-ones, and society-at-large.

Attention deficits are currently treated with stimulants, typically amphetamines. While such stimulants have been shown to improve focus, these prescription drugs are problematic. They come with significant side effects, such as poor sleep and weight loss, risks of abuse and addiction, and increased risk of early death (Dalsgaard et al. 2015). Notwithstanding, 70% of children and 40% of adults with a diagnosis of attention deficit will have stimulants prescribed (Burcu et al. 2016). The body typically adapts to stimulant drugs such as amphetamines, leading to the need for progressively higher doses. In addition, stimulants do not treat the other symptoms present in a subtype of ADHD that affects 30% of patients. A new approach is needed.

Pain syndromes, such as Fibromyalgia, are often non-specific and can affect individuals at any age, and the causes have not been clear. Identifying a homogeneous subgroup with an effective treatment would be a major advance.

Premenstrual Syndrome, a group of symptoms linked to the menstrual cycle, can affect menstruating women of any age between puberty and menopause and may present differently for each woman. Premenstrual Syndrome can exhibit a severity that fluctuates from a minor disturbance to a state in which it is difficult to get through the day, and can include cramps, migraines and other headaches. The causes of Premenstrual Syndrome are not clear.

Both, pain syndromes and Premenstrual Syndrome often lead to prescriptions of pain medications with significant addiction risk. A better approach to treatment is desirable.

### SUMMARY OF THE INVENTION

The invention features compositions and methods for the treatment of patients with Lidocaine-Ineffective Conditions. These treatments may also be useful for hypokalemic conditions. As described in detail below, in working with over 180 patients, I have found that patients with Lidocaine-Ineffective Conditions, including Sensory Overstimulation Syndrome (SOS), typically have a diagnosis of a disorder of attention, such as Attention Deficit Hyperactivity Disorder (ADHD), Attention Deficit Disorder (ADD), Asperger Syndrome, Sensory Processing Disorder, Sensory Integration Disorder, Fibromyalgia, various other pain disorders and/or Premenstrual Syndrome (PMS). Conditions amenable to the treatment paradigm described herein include these and others that fit the characteristics of Lidocaine-Ineffective Conditions. In certain embodiments of the methods of the invention, the patient may or may not have a comorbid neuropsychiatric condition. For example, women with Premenstrual Syndrome often do not have a comorbid neuropsychiatric condition. Without wishing to be bound by theory, I believe that such Lidocaine-Ineffective Conditions may be cause by one or more channelopathies or a transporter disease.

As described in detail below, I have discovered that such patients can be treated with potassium-related drugs. Compositions of matter and methods of use described herein relate to (1) Potassium supplementation, (a) alone, (b) in combination with drugs and herbal supplements used to treat ADHD, depression, anxiety, insomnia, or pain, or (c) with or without additional minerals, coatings for bioavailability, excipients, flavorings, or additions to improve stomach tolerance; and (2) Potassium-elevating agents, including renin-angiotensin-aldosterone inhibitors, such as those that are ineffective at reducing blood pressure, (a) alone; (b) combined with a blood pressure raising drug; or (c) combined with drugs and herbal supplements used to treat ADHD, depression, anxiety, insomnia, or pain, with or without additional minerals or coatings for bioavailability. Compositions may be in the form of a pill, tablet, powder, liquid or food conveyance. Compositions may be formulated as regular or extended-release, enabling less frequent administration, which has been shown in multiple studies to increase patient compliance significantly. Compositions can also be kitted (like is done with contraceptives) to improve compliance.

As described herein, the methods, compositions, and kits employ agents to elevate serum potassium in a subject in need thereof. One such agent is potassium, which can be administered in various salt forms, e.g., potassium gluconate, as is known in the art. It can be used alone or in combination with other therapeutic agents described below. In Lidocaine-Ineffective Conditions increasing the level of serum potassium is beneficial even in patients whose serum potassium is in the normal range. Another approach is to use potassium-elevating agents, such as renin/angiotensin/aldosterone inhibitors, alone or in combination with potassium or other therapeutic agents, for the conditions described below. I believe that elevating potassium levels using the methods and compositions of this invention, even among those whose serum potassium is in the normal range, may compensate for the underlying channelopathies or a transporter disease that may form the root cause of these Lidocaine-Ineffective Conditions.

The methods and compositions of this invention may also provide novel ways to treat classic conditions of hypokalemia, where the patient's serum potassium is below normal (<3.5 mEq/L), such as occurs as a side effect of chemotherapy.

In one aspect, the invention features a method of treating a Lidocaine-Ineffective Condition in a human patient by administering to the patient a therapeutically effective amount of potassium, directly, e.g., in a food conveyance or kit, or formulated for extended release with or without one or more additional therapeutic agents.

In another aspect, the invention features a method of treating a Lidocaine-Ineffective Condition in a human patient by administering to the patient a therapeutically effective amount of a potassium-elevating agent, directly, e.g., in a food conveyance or kit, or formulated for extended release with or without one or more additional therapeutic agents.

In a further aspect, the invention features a method of treating a Hypokalemic Condition in a human patient by administering to the patient a therapeutically effective amount of potassium, directly, e.g., in a food conveyance or kit, or formulated for extended release with or without one or more additional therapeutic agents.

In another aspect, the invention features a method of treating a Hypokalemic Condition in a human patient, the method comprising administering to the patient a potassium-elevating agent, directly, e.g., in a food conveyance or kit, or formulated for extended release with or without one or more additional therapeutic agents.

In another aspect, the invention features a composition of one of the above drugs, e.g., in the form of a food conveyance (e.g., powder, food, or beverage), including an amount of potassium or potassium-elevating agent, with or without an additional therapeutic agent, effective to treat a Lidocaine-Ineffective or Hypokalemic Condition.

In another aspect, the invention features kits to help effectively treat a Lidocaine-Ineffective or Hypokalemic Condition, with the components formulated separately or together (when more than one is present), including in the form of a food conveyance, into a plurality of dosage forms in an amount effective to treat a Lidocaine-Ineffective Disorder.

In some embodiments, prior to administering the treatment, the patient may be diagnosed as having partial or complete ineffectiveness of the anesthetic lidocaine. The patient may have or have been diagnosed as having a Lidocaine-Ineffective Condition (e.g., Sensory Overstimulation Syndrome, Attention Deficit Hyperactivity Disorder (ADHD), Attention Deficit Disorder (ADD), Asperger Syndrome, Sensory Processing Disorder, Sensory Integration Disorder, Fibromyalgia, various other pain disorders and/or Premenstrual Syndrome (PMS)), whether or not lidocaine effectiveness was tested. In certain embodiments of treating a Lidocaine-Ineffective Condition, the patient is not suffering from a hypokalemic condition such as Hypokalemic Periodic Paralysis (HPP). The administering may be oral, subdermal, ocular, otic, vaginal, rectal, IV, intranasal, transdermal, or other routes as described herein. In some embodiments of the methods, compositions, and kits, the potassium may include a potassium salt (e.g., potassium gluconate and potassium chloride). The potassium may be formulated in a pill, tablet, capsule, powder, liquid, or food conveyance. In some embodiments, the potassium is administered to the human patient in multiple doses, from 1 to 30 dosage forms (e.g., pills, tablets, or food conveyances), such as in 1 to 24, 2 to 20, 2 to 15, 2 to 12, 2 to 9, 3 to 8, 2 to 7, or 3 to 6 dosage forms (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 dosage forms). A total of elemental potassium, for example, from 90 mg to 5000 mg, e.g., 90 to 1000 mg, 250 to 4000 mg, 500 to 4000 mg, 750 to 4000 mg, 1000 to 4000 mg, 1250 to 4000, 1500 to 4000 mg. 2000 to 4000 mg, 1000 to 2000 mg, 1000 to 3000, or 3000 to 5000 mg, may be administered to the patient per day (e.g., about 90 mg, 100 mg, 125 mg, 150 mg, 175 mg, 200 mg, 225 mg, 250 mg, 500 mg, 750 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg, 3,000 mg, 3,500 mg, 4,000 mg, 4,500 mg, or 5,000 mg of elemental potassium per day). For example, a child may be administered from about 250 mg per dose of elemental potassium, and an adult may be administered from about 500 mg per dose of elemental potassium, and may take 4-5 such doses in 24 hours. Further dosages and ranges are provided herein. The potassium may be packaged in a kit. The potassium may be formulated for extended release. In certain embodiments, individual dosage forms, e.g., a food conveyance, includes a total of 200 mg or more of elemental potassium, e.g., 200-800 mg, 300-600 mg, or 400-600 mg.

In some embodiments, the patient being treated has a serum potassium level between 3.5-5.0, 3.5-4.5, 3.5-4.0, 3.5-3.75, or 3.5-3.6 mEq/L, either on average or at the time of administration.

The methods may employ acute or chronic administration. For example, the patient may be treated once or a few times in a limited period of time or treated continually over a period of one week, two weeks, one month, three months, six months, one year, or longer. Patients being treated chronically may also be treated acutely with additional doses as needed.

In some embodiments, an additional therapeutic agent for a Lidocaine-Ineffective Condition is useful in a method, composition, or kit of the invention. The additional therapeutic agent may be a TAAR1 agonist (e.g., amphetamine, levoamphetamine, dextroamphetamine, and lisdexamfetamine), an inhibitor of neurotransmitter reuptake of one or more of norepinephrine, dopamine, and serotonin (e.g., methylphenidate, dexmethylphenidate, atomexetine, modafinil, armodafinil, bupropion, and venlafaxine), an alpha-2 adrenergic receptor agonist (e.g., clonidine and guanfacine), a monoamine oxidase inhibitor (e.g., selegiline, tranylcypromine, and phenelzine), an adenosine receptor antagonist (e.g., caffeine, theophylline, and theobromine), a barbiturate (e.g., secobarbital, pentobarbital, phenobarbital, amobarbital, and butabarbital); a benzodiazepine (e.g., alprazolam, diazepam, lorazepam, temazepam, clonazepam, oxazepam, quazepam, flurazepam, adinazolam, estazolam, flubromazolam, nitrazolam, pyrazolam, triazolam, and zapizolam); a hypnotic agent (e.g., chloral hydrate, eszopiclone, tasimelteon, zolpidem, ramelteon, SAR, melatonin, agomelatine, tasimelteon, TIK-301, and suvorexant); an antihistamine (e.g., acrivastine, azelastine, acrivastine, cetirizine, diphenhydramine, bilastine, bromodiphenhydramine, brompheniramine, buclizine, carbinoxamine, chlorodiphenhydramine, chlorphenamine, chlorpromazine, clemastine, cyclizine, cyproheptadine, dexbrompheniramine, dexchlorpheniramine, dimenhydrinate, dimetindene, doxylamine, ebastine, embramine, fexofenadine, loratidine, hydroxyzine, meclizine, mirtazapine, olopatadine, orphenadrine, phenindamine, pheniramine, phenyltoloxamine, promethazine, rupatadine, tripelennamine, and triprolidine), a pyrazolopyrimidine (e.g., zaleplon, indiplon, ocinaplon, divaplon, and lorediplon); a serotonin antagonist and reuptake inhibitor (SARI) (e.g., trazodone, nefazodone, mepiprazole, lubazodone, loriprazole, and etoperidone); a selective serotonin reuptake inhibitor (SSRI) (e.g., sertraline, escitalopram, fluoxetine, citalopram, and paroxetine); a beta blocker (e.g., propranolol and atenolol); a serotonin-norepinephrine reuptake inhibitor (SNRI) (e.g., duloxetine, venlafaxine, desvenlafaxine, atomozetine, milnacipran, and levomilnacipran); a tricyclic antidepressant (TCA) (e.g., nortriptyline, imipramine, amoxapine, desipramine, dibenzocycloheptadiene, trimipramine, doxepin, amitriptyline/chlordiazepoxide, clomipramine, amitriptyline/perphenazine, and protriptyline); a tetracyclic antidepressant (e.g., mirtazapine, maprotiline, and a piperazino-azepine); an antipsychotic (e.g., aripiprazole, olanzapine, risperidone, paliperidone, and brexipiprazole); an opioid (e.g., codeine, morphine, thebaine, oripavine, diacetylmorphine, nicomorphine, dipropanoylmorphine, diacetyldihydromorphine, acetylpropionylmorphine, desomorphine, methyldesorphine, dibenzoylmorphine, dihydrocodeine, ethylmorphine, heterocodeine, buprenorphine, etorphine, hydrocodone, hydromorphone, oxycodone, oxymorphone, fentanyl, alphamethylfentanyl, alfentanil, sufentanil, remifentanil, carfentanyl, ohmefentanyl, pethidine, ketobemidone, mppp, allylprodine, prodine, pepap, promedol, propoxyphene, dextropropoxyphene, dextromoramide, bezitramide, piritramide, methadone, dipipanone, levomethadyl acetate, difenoxin, diphenoxylate, loperamide, dezocine, pentazocine, phenazocine, buprenorphine, dihydroetorphine, etorphine, butorphanol, nalbuphine, levorphanol, levomethorphan, racemethorphan, lefetamine, menthol, meptazinol, mitragynine, tilidine, tramadol, tapentadol, eluxadoline, AP-237, and 7-hydroxymitragynine); a folate treatment (e.g., vitamin B12 and folic acid); a treatment for mania (e.g., lithium, quetiapine, and valproate); a serotonin modulator and stimulator (SMS) (e.g., vilazodone and vortioxetine); a vitamin B3 complex component (e.g., nicotinic acid (niacin) and nicotinamide (niacinamide)); a treatment for hypothyroidism (e.g., desiccated thyroid); a muscle relaxant (e.g., cyclobenzaprine and tizanidine); an anticonvulsant (e.g., lamotrigine, pregabalin, and gabapentin); a diuretic (e.g., a thiazide-based diuretic (e.g., indapamide, hydrochlorothiazide, chlorthalidone, chlorothiazide, metolazone, methyclothiazide, bendroflumethiazide, polythiazide, and hydroflumethiazide), a loop-based diuretic (e.g., bumetanide, thacrynic acid, torsemide, and ethacrynic acid), a potassium-sparing diuretic (e.g., triamterene; spironolactone, and amiloride), pamabrom, and mannitol); or a stomach medicine (e.g., bismuth subsalicylate, calcium carbonate, and ranitidine). The potassium-sparing diuretic may be administered with a thiazide (e.g., hydrochlorothiazide). The additional therapeutic agent may also be acontium napellus, chocolate, cinchona officinalis, coffee, gnaphalium polycephalum, guarana, guayusa, leduum palustre, magnesia phosphorica, rhus toxicodendron, tea, viscum album, Hypericum, yaupon, and khat. The additional therapeutic agent may also be a nonsteroidal anti-inflammatory drug (NSAID) (e.g., aspirin, diclofenac, diflunisal, indomethacin, sulindac, etodolac, mefenamic acid, meclofenamate, flufenamic acid, tolmetin, ketorolac, diclofenac, ibuprofen, naproxen, fenoprofen, ketoprofen, flurbiprofen, oxaprozin, piroxicam, meloxicam, nabumetone, celecoxib, valdecoxib, parecoxib, etoricoxib, and lumaricoxib). An antihistamine may be formulated, kitted, or use in combination with a nonsteroidal anti-inflammatory drug (NSAID).

In certain embodiments of the methods, compositions, and kits, the potassium-elevating agent may be formulated in a pill, tablet, capsule, powder, liquid, or food conveyance. The potassium-elevating agent may be a renin-angiotensin-aldosterone system antagonist, or another drug that elevates serum potassium. The potassium-elevating agent, e.g., renin-angiotensin-aldosterone system antagonist, may be administered at a dose of from 0.05 mg/day to 600 mg/day, e.g., 0.05 to 50, 10 to 100, 10 to 200, 10 to 300, 100 to 500, 100 to 400, 100 to 300, 200 to 600, or 300 to 600 mg/day. Further dosages and ranges are provided herein (e.g., about 0.05 mg/day, 10 mg/day, 20 mg/day, 30 mg/day, 40 mg/day, 50 mg/day, 60 mg/day, 70 mg/day, 80 mg/day, 90 mg/day, 100 mg/day, 200 mg/day, 300 mg/day, 400 mg/day, 500 mg/day, or 600 mg/day). The potassium-elevating agent, e.g., renin-angiotensin-aldosterone system antagonist, may be administered to the patient in 1 to 30 dosage forms, such as in 1 to 24, 2 to 20, 2 to 15, 2 to 12, 2 to 9, 3 to 8, 2 to 7, or 3 to 6 dosage forms (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30). The potassium-elevating agent may be packaged in a kit. The potassium-elevating agent may be formulated for extended release. A drug that increases blood pressure by a mechanism other than the renin-angiotensin-aldosterone system (e.g., fludrocortisone and midodrine) may be useful in a method, composition, or kit described herein.

The renin-angiotensin-aldosterone system antagonist may be an ACE inhibitor (e.g., captopril, zofenopril, enalapril, ramipril, quinapril, perindopril, lisinopril, benazepril, imidapril, trandolapril, cilazapril, fosinopril, moexipril, spirapril, alacepril, deparil, temocapril, and teprotide), an angiotensin receptor antagonist (e.g., of losartan, candesartan, valsartan, irbesartan, telmisartan, eprosartan, olmesartan, azilsartan, and firmasartan), an aldosterone antagonist (e.g., spironolactone and eplerenone), or a renin inhibitor (e.g., aliskiren). In some embodiments, the renin-angiotensin-aldosterone system antagonist does not substantially reduce blood pressure.

In some embodiments of the methods, compositions, and kits above, the potassium, potassium elevating agent, composition, and/or kit may be in the form of a consumable form and/or food conveyance (e.g., powder, food, and beverage). The food conveyance may be kitted or administered to the patient in 1 to 30 dosage forms, as described herein. The consumable form may be packaged in a kit. The dosages may be formulated as food conveyances.

In some embodiments, the methods, compositions, or kits do not include a combination of a renin-angiotensin-aldosterone system antagonist and a stimulant or MAO inhibitor, e.g., for use with a patient diagnosed with ADHD. In some embodiments, the methods, compositions, or kits do not include a combination of a serotonin receptor modulator and a renin-angiotensin-aldosterone system antagonist, e.g., for use with a patient diagnosed with ADHD. In some embodiments, the methods, compositions, or kits do not include a combination of a dopamine reuptake inhibitor and a renin-angiotensin-aldosterone system antagonist, e.g., for use with a patient diagnosed with ADHD. In some embodiments, the methods, compositions, or kits do not include a combination of caffeine and a renin-angiotensin-aldosterone system antagonist, e.g., for use with a patient diagnosed with ADHD. In some embodiments, the methods, compositions, or kits do not include a renin inhibitor (e.g., aliskiren) or a combination of a renin inhibitor and methylphenidate, e.g., for use with a patient diagnosed with ADHD. In some embodiments, the methods, compositions, or kits do not include a combination of potassium and methylphenidate, e.g., for use with a patient diagnosed with chronic fatigue, such as that comorbid with ADD or ADHD. In some embodiments, the methods, compositions, or kits do not include a combination of an adenosine receptor antagonist and a renin-angiotensin-aldosterone system antagonist, e.g., for use with a patient diagnosed with ADHD. In some embodiments, the methods, compositions, or kits do not include an ACE inhibitor (e.g., captopril), e.g., for use with a patient diagnosed with ADHD. In some embodiments, the methods, compositions, or kits do not include a combination of methylphenidate and an angiotensin II receptor antagonist (e.g., losartan), e.g., for use with a patient diagnosed with ADHD.

In another aspect, the invention features a method of treating a Lidocaine-Ineffective Condition in a human patient by administering to the patient a therapeutically effective amount of a renin-angiotensin-aldosterone system antagonist that boosts potassium. The patient can be one that has been diagnosed as having a Lidocaine-Ineffective Condition, whether or not lidocaine effectiveness was tested. In some embodiments, the method further includes diagnosing the patient as having a lidocaine ineffectiveness prior to the administering.

In another aspect, the invention features a method of treating a Lidocaine-Ineffective Condition in a human patient by administering to the patient a therapeutically effective amount of (A) a renin-angiotensin-aldosterone system antagonist that boosts potassium in combination with (B) a drug that increases blood pressure by another mechanism, to extend the effectiveness of this therapy to those with normal and low blood pressure, who would otherwise become hypotensive with treatment. The patient can be one that has been diagnosed as having a Lidocaine-Ineffective Condition, whether or not lidocaine effectiveness was tested. In some embodiments, the method further includes diagnosing the patient as having a lidocaine ineffectiveness prior to the administering.

In another aspect, the invention features a method of treating a Lidocaine-Ineffective Condition in a human patient by administering to the patient a therapeutically effective amount of (A) a non-potassium ADHD treatment, as described in more detail herein, and (B) a renin-angiotensin-aldosterone system antagonist that boosts potassium. The patient can be one that has been diagnosed as having a Lidocaine-Ineffective Condition, whether or not lidocaine effectiveness was tested. In some embodiments, the method further includes diagnosing the patient as having a lidocaine ineffectiveness prior to the administering. This method can further include administering a drug that increases blood pressure by a mechanism other than the renin-angiotensin-aldosterone system.

In another aspect, the invention features a method of treating a Lidocaine-Ineffective Condition in a human patient by administering to the patient a therapeutically effective amount of (A) a non-potassium ADHD treatment and (B) a potassium salt. The patient can be one that has been diagnosed as having a Lidocaine-Ineffective Condition, whether or not lidocaine effectiveness was tested. In some embodiments, the method further includes diagnosing the patient as having a lidocaine ineffectiveness prior to the administering.

In another aspect, the invention features a method of treating premenstrual syndrome in a human patient by administering to the patient a therapeutically effective amount of an adenosine receptor antagonist and a renin-angiotensin-aldosterone system antagonist, wherein the patient has previously been diagnosed as having a Lidocaine-Ineffective Condition, whether or not lidocaine effectiveness was tested.

In another aspect, the invention features a method of treating premenstrual syndrome in a human patient by administering to the patient a therapeutically effective amount of an adenosine receptor antagonist and potassium.

In another aspect, the invention features a method of treating Asperger syndrome in a human patient by administering to the patient a therapeutically effective amount of risperidone and a renin-angiotensin-aldosterone system antagonist or potassium.

In another aspect, the invention features a method of treating a Lidocaine-Ineffective Condition in a human patient by administering to the patient a therapeutically effective amount of a renin-angiotensin-aldosterone system antagonist.

The invention also features pharmaceutical compositions and kits that includes a therapeutically effective amount of a non-potassium ADHD treatment and either a renin-angiotensin-aldosterone system antagonist (with or without a drug that increases blood pressure by a mechanism other than the renin-angiotensin-aldosterone system) or potassium salt. Other pharmaceutical compositions and kits include a renin-angiotensin-aldosterone system antagonist and a drug that increases blood pressure by another mechanism. In pharmaceutical compositions, the agents, e.g., the non-potassium ADHD treatment and either a renin-angiotensin-aldosterone system antagonist or potassium salt, are formulated in a single dosage form (e.g., pill or tablet). In the kits of the invention, the active agents may or may not be formulated together. When formulated together for at least one dosage, the kit will include multiple dosages. In one embodiment, when the non-potassium ADHD treatment is a stimulant, the kit includes fewer doses of the simulant relative to the renin-angiotensin-aldosterone system antagonist or potassium. For example, the kit may include paired doses of a non-potassium ADHD treatment and the renin-angiotensin-aldosterone system antagonist or potassium, except for one additional dose of the renin-angiotensin-aldosterone system antagonist or potassium intended to be taken before bed time. Alternatively, the kit may include multiple doses of the combined agents with an additional dose, intended for administration prior to bed time, including the renin-angiotensin-aldosterone system antagonist or potassium but not the stimulant. The dose not including the stimulant may have a different appearance, shape, or form to distinguish.

In one aspect, the pharmaceutical composition may include (A) a non-potassium ADHD treatment, and (B) a renin-angiotensin-aldosterone system antagonist in amounts effective to treat a Lidocaine-Ineffective Condition. In another aspect, the pharmaceutical composition may include (A) a non-potassium ADHD treatment, and (B) potassium in amounts effective to treat a Lidocaine-Ineffective Condition. In yet another aspect, the pharmaceutical composition may include a renin-angiotensin-aldosterone system antagonist and a drug that increases blood pressure by a mechanism other than the renin-angiotensin-aldosterone system.

In one aspect, the kit may include a renin-angiotensin-aldosterone system antagonist and a drug that increases blood pressure by a mechanism other than the renin-angiotensin-aldosterone system. In another aspect, the kit may include (A) a non-potassium ADHD treatment, and (B) potassium in amounts effective to treat a Lidocaine-Ineffective Condition.

The non-potassium ADHD treatments used can vary widely. In some embodiments, the treatment is:
(a) A TAAR1 agonist, such as amphetamine, levoamphetamine, dextroamphetamine, or lisdexamfetamine; or
(b) An inhibitor of reuptake of one or more of norepinephrine, dopamine, and serotonin, such as methylphenidate, dexmethylphenidate, atomexetine, modafinil, bupropion, and venlafaxine; or
(c) An alpha-2 adrenergic receptor agonist, such as clonidine (as HCl salt or free base or alone or in combination with chlorthalidone) and guanfacine; or
(d) A monoamine oxidase inhibitor, such as selegiline; or
(e) An adenosine receptor antagonist, such as caffeine, theophylline, and theobromine; or
(f) An additional agent, such as carbamazepine, pemoline, risperidone, and metadoxine.
Certain of these non-potassium ADHD treatments are also classified as stimulants, as is known in the art.

The renin-angiotensin-aldosterone system antagonist used can vary widely. In some embodiments, the renin-angiotensin-aldosterone system antagonist is:
(a) An angiotensin converting enzyme (ACE) inhibitor, such as captopril, zofenopril, enalapril, ramipril, quinapril, perindopril, lisinopril, benazepril, imidapril, trandolapril, cilazapril, fosinopril, moexipril, spirapril, alacepril, deparil, temocapril, and teprotide; or
(b) An angiotensin receptor antagonist, such as losartan, candesartan, valsartan, irbesartan, telmisartan, eprosartan, olmesartan, azilsartan, and firmasartan; or
(c) An aldosterone antagonist, such as spironolactone (alone or in combination with hydrochlorothiazide) and Eplerenone; or
(d) A renin inhibitor, such as aliskiren.

In some embodiments, the drug that increases blood pressure, e.g., fludrocortisone or midodrine, is added.

In some embodiments, the potassium salt is potassium gluconate.

The potassium dose over a 24-hours will vary by age and size of patient. An adult will typically take 4-5 doses of 600 mg of elemental potassium; children will typically take doses of half that amount.

In some embodiments, the potassium is administered to the human patient in multiple doses, from 1 to 10 dosage forms (e.g., pills or tablets), such as in 2 to 9 dosage forms, 3 to 8 dosage forms, 2 to 7 dosage forms, or 3 to 6 dosage forms. A total of, for example, from about 250 to about 5,000 mg of the potassium may be administered to the patient per day (e.g., about 250, 500 mg, 750 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg, 3,000 mg, 3,500 mg, 4,000 mg, 4,500 mg, or 5,000 mg of potassium per day). For example, a child may be administered from about 250 mg per day, and an adult may be administered from about 500 mg per day.

In some embodiments, the potassium is administered using a time release form to deliver the above doses in a steady amount throughout a longer time period.

In some embodiments, the ratio of doses of the components can be varied over time. For example, when used in combination with stimulants, the stimulants can be designed to taper to allow sleep, while the potassium can be maintained at the same level, including during sleep.

In some embodiments, the method additionally includes administering to the patient a therapeutically effective amount of a substance selected from the group consisting of acontium napellus, cinchona officinalis, gnaphalium polycephalum, leduum palustre, magnesia phosphorica, rhus toxicodendron, viscum album, Hypericum, and khat. Additionally or alternatively, the method may include administering to the patient a therapeutically effective amount of a nonsteroidal anti-inflammatory drug (NSAID), such as aspirin.

In another aspect, the invention features a method of treating a Lidocaine-Ineffective Condition in a human patient by administering to the patient a therapeutically effective amount of a renin-angiotensin-aldosterone system antagonist that boosts potassium. The patient can be one that has been diagnosed as having a Lidocaine-Ineffective Condition, whether or not lidocaine effectiveness was tested. In some embodiments, the method further includes diagnosing the patient as having a lidocaine ineffectiveness prior to the administering.

In another aspect, the invention features a method of treating a Lidocaine-Ineffective Condition in a human patient by administering to the patient a therapeutically effective amount of (A) a renin-angiotensin-aldosterone system antagonist that boosts potassium in combination with (B) a drug that increases blood pressure by another mechanism, to extend the effectiveness of this therapy to those with normal and low blood pressure, who would otherwise become hypotensive with treatment. The patient can be one that has been diagnosed as having a Lidocaine-Ineffective Condition, whether or not lidocaine effectiveness was tested. In some embodiments, the method further includes diagnosing the patient as having a lidocaine ineffectiveness prior to the administering.

In another aspect, the invention features a method of treating a Lidocaine-Ineffective Condition in a human patient by administering to the patient a therapeutically effective amount of (A) a non-potassium ADHD treatment, as described in more detail herein, and (B) a renin-angiotensin-aldosterone system antagonist that boosts potassium. The patient can be one that has been diagnosed as having a Lidocaine-Ineffective Condition, whether or not lidocaine effectiveness was tested. In some embodiments, the method further includes diagnosing the patient as having a lidocaine ineffectiveness prior to the administering. This method can further include administering a drug that increases blood pressure by a mechanism other than the renin-angiotensin-aldosterone system.

In another aspect, the invention features a method of treating a Lidocaine-Ineffective Condition in a human patient by administering to the patient a therapeutically effective amount of (A) a non-potassium ADHD treatment and (B) a potassium salt. The patient can be one that has been diagnosed as having a Lidocaine-Ineffective Condition, whether or not lidocaine effectiveness was tested. In some embodiments, the method further includes diagnosing the patient as having a lidocaine ineffectiveness prior to the administering.

In another aspect, the invention features method of treating Asperger syndrome in a human patient by administering to the patient a therapeutically effective amount of risperidone and a renin-angiotensin-aldosterone system antagonist or potassium.

In another aspect, the invention features a method of treating premenstrual syndrome in a human patient by administering to the patient a therapeutically effective amount of an adenosine receptor antagonist and a renin-angiotensin-aldosterone system antagonist, wherein the patient has previously been diagnosed as having a Lidocaine-Ineffective Condition, whether or not lidocaine effectiveness was tested.

In another aspect, the invention features a method of treating premenstrual syndrome in a human patient by administering to the patient a therapeutically effective amount of an adenosine receptor antagonist and potassium.

The invention also features pharmaceutical compositions and kits that includes a therapeutically effective amount of a non-potassium ADHD treatment and either a renin-angiotensin-aldosterone system antagonist (with or without a drug that increases blood pressure by a mechanism other than the renin-angiotensin-aldosterone system) or potassium salt. Other pharmaceutical compositions and kits include a renin-angiotensin-aldosterone system antagonist and a drug that increases blood pressure by another mechanism. In pharmaceutical compositions, the agents, e.g., the non-potassium ADHD treatment and either a renin-angiotensin-aldosterone system antagonist or potassium salt, are formulated in a single dosage form (e.g., pill or tablet). A pharmaceutical composition can be delivered in traditional form, e.g., pill or liquid form or in a food or beverage formulation that accommodates the bulk of potassium salts. In the kits of the invention, the active agents may or may not be formulated together. When formulated together for at least one dosage, the kit will include multiple dosages. In one embodiment, when the non-potassium ADHD treatment is a stimulant, the kit includes fewer doses of the simulant relative to the renin-angiotensin-aldosterone system antagonist or potassium. For example, the kit may include paired doses of a non-potassium ADHD treatment and the renin-angiotensin-aldosterone system antagonist or potassium, except for one additional dose of the renin-angiotensin-aldosterone system antagonist or potassium intended to be taken before bed time. Alternatively, the kit may include multiple doses of the combined agents with an additional dose, intended for administration prior to bed time, including the renin-angiotensin-aldosterone system antagonist or potassium but not the stimulant. The dose not including the stimulant may have a different appearance, shape, or form to distinguish.

In another aspect, the invention features a pharmaceutical composition including (A) a non-potassium ADHD treatment, and (B) a renin-angiotensin-aldosterone system antagonist in amounts effective to treat a Lidocaine-Ineffective Condition.

In another aspect, the invention features a pharmaceutical composition including (A) a non-potassium ADHD treatment, and (B) potassium in amounts effective to treat a Lidocaine-Ineffective Condition.

In another aspect, the invention features a pharmaceutical composition including a renin-angiotensin-aldosterone system antagonist and a drug that increases blood pressure by a mechanism other than the renin-angiotensin-aldosterone system.

In another aspect, the invention features a kit including (A) a non-potassium ADHD treatment, and (B) a renin-angiotensin-aldosterone system antagonist in amounts effective to treat a Lidocaine-Ineffective Condition.

In another aspect, the invention features a kit including (A) a non-potassium ADHD treatment, and (B) potassium in amounts effective to treat a Lidocaine-Ineffective Condition.

In another aspect, the invention features a kit including a renin-angiotensin-aldosterone system antagonist and a drug that increases blood pressure by a mechanism other than the renin-angiotensin-aldosterone system

The non-potassium ADHD treatments used can vary widely. In some embodiments, the treatment is:
(a) A TAAR1 agonist, such as amphetamine, levoamphetamine, dextroamphetamine, or lisdexamfetamine; or
(b) An inhibitor of reuptake of one or more of norepinephrine, dopamine, and serotonin, such as methylphenidate, dexmethylphenidate, atomexetine, modafinil, bupropion, and venlafaxine; or
(c) An alpha-2 adrenergic receptor agonist, such as clonidine (as HCl salt or free base or alone or in combination with chlorthalidone) and guanfacine; or
(d) A monoamine oxidase inhibitor, such as Selegiline; or
(e) An adenosine receptor antagonist, such as caffeine, theophylline, and theobromine; or
(f) An additional agent, such as carbamazepine, pemoline, risperidone, and metadoxine.
Certain of these non-potassium ADHD treatments are also classified as stimulants, as is known in the art.

The renin-angiotensin-aldosterone system antagonist used can vary widely. In some embodiments, the renin-angiotensin-aldosterone system antagonist is:
(a) An angiotensin converting enzyme (ACE) inhibitor, such as captopril, zofenopril, enalapril, ramipril, quinapril, perindopril, lisinopril, benazepril, imidapril, trandolapril, cilazapril, fosinopril, moexipril, spirapril, alacepril, deparil, temocapril, and teprotide; or
(b) An angiotensin receptor antagonist, such as losartan, candesartan, valsartan, irbesartan, telmisartan, eprosartan, olmesartan, azilsartan, and firmasartan; or
(c) An aldosterone antagonist, such as spironolactone (alone or in combination with hydrochlorothiazide) and Eplerenone; or
(d) A renin inhibitor, such as aliskiren.

In some embodiments, the drug that increases blood pressure, e.g., fludrocortisone or midodrine, is added.

In some embodiments, the potassium salt is potassium gluconate.

The potassium dose over a 24-hours will vary by age and size of patient. An adult will typically take 4-5 doses of 600 mg of elemental potassium; children will typically take doses of half that amount.

In some embodiments, the potassium is administered to the human patient in multiple doses, from 1 to 10 dosage forms (e.g., pills or tablets), such as in 2 to 9 dosage forms, 3 to 8 dosage forms, 2 to 7 dosage forms, or 3 to 6 dosage forms. A total of, for example, from about 250 to about 5,000 mg of the potassium may be administered to the patient per day (e.g., about 250, 500 mg, 750 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg, 3,000 mg, 3,500 mg, 4,000 mg, 4,500 mg, or 5,000 mg of potassium per day). For example, a child may be administered from about 250 mg per day, and an adult may be administered from about 500 mg per day.

In some embodiments, the potassium is administered using a time release form to deliver the above doses in a steady amount throughout a longer time period.

In some embodiments, the ratio of doses of the components can be varied over time. For example, when used in combination with stimulants, the stimulants can be designed to taper to allow sleep, while the potassium can be maintained at the same level, including during sleep.

In some embodiments, the method additionally includes administering to the patient a therapeutically effective amount of a substance selected from the group consisting of acontium napellus, chocolate, cinchona officinalis, coffee, gnaphalium polycephalum, guarana, guayusa, leduum palustre, magnesia phosphorica, rhus toxicodendron, tea, viscum album, Hypericum, yaupon, and khat. Additionally or alternatively, the method may include administering to the patient a therapeutically effective amount of a nonsteroidal anti-inflammatory drug (NSAID), such as aspirin.

### Definitions

As used herein, the term “about” refers to a value that is within 10% above or below the value being described.

As used herein, the term “aldosterone antagonist” refers to a compound having the ability to counteract the effect of aldosterone, for example, by competitive blockage aldosterone receptors found in renal tubules. Aldosterone antagonists useful in conjunction with the compositions and methods described herein include those known in the art, such as those described in US Patent Application Publication No. 2006/0286105, the disclosure of which is incorporated herein by reference as it pertains to aldosterone antagonists.

As used herein, the term “angiotensin-converting enzyme inhibitor” or “ACE inhibitor” refers to a substance having the ability to inhibit the cleavage of the N-terminal decapeptide angiotensin I to the vasoactive octapeptide angiotensin II. ACE inhibitors useful in conjunction with the compositions and methods described herein include those known in the art, such as those described in, for example, U.S. Pat. Nos. 4,046,889 and 4,374,829, the disclosures of each of which are incorporated herein by reference as they pertain to ACE inhibitors.

As used herein, the term “angiotensin receptor antagonist” refers to a compound having the ability to inhibit the vasoactive effects of endogenous angiotensin II by competitive blockade at the angiotensin receptor sites located in vascular smooth muscle and within the adrenal gland. Angiotensin receptor antagonists include compounds capable of binding the angiotensin receptor as well as those capable of binding angiotensin II to compete with or otherwise preclude interaction between angiotensin II and the angiotensin II receptor. Angiotensin receptor antagonists useful in conjunction with the compositions and methods described herein include those known in the art, such as those described in, for example, U.S. Pat. Nos. 4,355,040 and 4,880,804, the disclosures of each of which are incorporated herein by reference as they pertain to angiotensin receptor antagonists.

As used herein, the term “disorder of attention” refers to a condition characterized by inattention, over-activity, and/or impulsiveness. Disorders of attention include, without limitation, Attention Deficit Hyperactivity Disorder, Attention Deficit Disorder, Hyperkinetic Disorder, Sensory Processing Disorder, Sensory Integration Disorder, Sensory Overstimulation Syndrome (SOS), Hypokalemic Sensory Overstimulation and Premenstrual Syndrome (PMS). Attention Deficit Hyperactivity Disorder, which is also referred to in the literature as Attention Deficit Disorder/Hyperactivity Syndrome (ADD/HS), is a condition (or group of conditions) characterized by impulsiveness, distractibility, inappropriate behavior in social situations and hyperactivity. Other disorders, such as Asperger Syndrome, may include a finding of attention deficit and are included in this definition.

As used herein, the term “food conveyance” is a substance that can be consumed for nutrition. For example, a food conveyance can be a food or beverage.

As used herein, the term “Lidocaine-Ineffective Condition” is a condition in a patient on whom lidocaine is ineffective as an anesthetic. In such disorders I have found that the condition is improved by increasing potassium. As described above, a patient with a Lidocaine-Ineffective Condition may currently have a diagnosis of a disorder of attention such as Sensory Overstimulation Syndrome (SOS), Attention Deficit Hyperactivity Disorder (ADHD), Attention Deficit Disorder (ADD), Asperger Syndrome, Sensory Processing Disorder, Sensory Integration Disorder, Fibromyalgia, various other pain disorders and/or Premenstrual Syndrome (PMS).

As used herein, the term “Hypokalemia” means low serum potassium, as defined as below 3.5 mEq/L.

As used herein, the term “hypokalemic condition” refers to a condition characterized by or exacerbated by serum potassium falling into the hypokalemic range. A patient with a hypokalemic condition will have potassium serum concentration low during exacerbation of the condition or all the time in certain conditions. Examples of hypokalemic conditions include Bartter syndrome.

As used herein, the term “nonsteroidal anti-inflammatory drug” or “NSAID” refers to a nonsteroidal compound that exhibits anti-inflammatory, antipyretic, and analgesic properties. Examples of NSAIDs include those described herein and known in the art, such as those described in U.S. Pat. No. 4,985,459, the disclosure of which is incorporated herein by reference as it pertains to NSAIDs. For a detailed account of the chemical structures, syntheses, and pharmacological properties of NSAIDs, see Anti-Inflammatory and Anti-Rheumatic Drugs. K. D. Rainsford, Vol. I-III, CRC Press, Boca Raton (1985), and Anti-Inflammatory Agents. Chemistry and Pharmacology, 1 R. A. Scherrer, et al., Academic Press, New York (1974), the disclosures of each of which are incorporated herein by reference as they pertain to NSAIDs.

As used herein, the term “pharmaceutical composition” means a mixture containing a therapeutic compound to be administered to a subject, such as a mammal, e.g., a human, and a carrier to prevent, treat, or control a particular disease or condition affecting the mammal or to meet the distinctive dietary requirements of the condition.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “potassium elevating agent” refers to a substance that increases the serum concentration of potassium by a mechanism other than direct administration of potassium. Examples are provided herein.

As used herein, the term “Premenstrual Syndrome” refers to a combination of physical and emotional disturbances that occur after a woman ovulates and ends with or shortly after menstruation.

As used herein, the term “renin inhibitor” refers to a substance capable of inhibiting the initial, rate-limiting step in the renin-angiotensin system cascade: renin-mediated proteolytic conversion of angiotensinogen into the N-terminal decapeptide angiotensin I, the penultimate precursor to angiotensin II. Renin inhibitors include compounds that specifically bind renin, such as compounds that bind the proteolytic active site of renin to preclude the binding and subsequent cleavage of angiotensin. Renin inhibitors useful in conjunction with the compositions and methods described herein include those known in the art, such as those described in, for example, U.S. Pat. Nos. 4,814,342; 4,855,303; and 4,895,834, the disclosures of each of which are incorporated herein by reference as they pertain to renin inhibitors.

As used herein, the term “Sensory Overstimulation Syndrome” refers to a condition that can present with findings of inattention, pain, cramps, migraines, or Premenstrual Syndrome (in females), as well as being lidocaine insensitive. During episodes of overstimulation, patients will find that small stimuli will produce outsized reactions. For example, sounds will seem louder (and even hostile), clothing will seem intolerably irritating and visual cues will be completely distracting. The syndrome is believed to be the result of a channelopathy affecting the sensory nerves.

As used herein, the terms “subject” and “patient” are interchangeable and refer to an organism that receives treatment for a particular disease or condition as described herein or that is diagnosed as having a disease or condition according to the methods described herein. Such subject or patient can be a mammal, including a human.

As used herein, the term “therapeutically effective” refers to an amount of a therapeutic agent sufficient to result in prevention, delay of onset, and/or amelioration of one or more symptoms of a Lidocaine-Ineffective Condition or hypokalemic condition.

As used herein, the terms “treat” or “treatment” refer to therapeutic treatment, in which the object is to alleviation or amelioration of one or more symptoms or conditions; diminishment of extent of disease, disorder, or condition; stabilization (i.e., not worsening) of a state of disease, disorder, or condition; delay or slowing the progress of the disease, disorder, or condition; amelioration or palliation of the disease, disorder, or condition; and remission (whether partial or total), whether detectable or undetectable. In some examples, treating also includes meeting the distinct dietary requirements of a condition.

### DETAILED DESCRIPTION

Through our work with over 180 patients, I have surprisingly discovered a genetic syndrome believed to be autosomal-dominant that appears to underlie symptoms in millions of Americans who currently have a diagnosis of Attention Deficit Hyperactivity Disorder (ADHD) and/or Premenstrual Syndrome (PMS), or Asperger Syndrome, Sensory Processing Disorder, Sensory Integration Disorder, Fibromyalgia, and various other pain disorders (Segal et al. 2007, Segal 2014). I call this condition “Sensory Overstimulation Syndrome” (SOS), believed to be the cause of the symptoms of a sub-group with each of the diagnoses listed.

A distinctive feature of SOS is relative ineffectiveness of the local anesthetic lidocaine, a drug that blocks sodium channels. I provide a novel approach that uses potassium-related treatments to treat these disorders and optionally lidocaine to test for these disorders. Lidocaine is an anesthetic used to numb tissues. It works by blocking the sodium channel in the sensory neurons. However, for about 2.7-11% of the population, lidocaine is ineffective. (Rozanski et al. 1988, Nakai et al. 2000). People with relative insensitivity to lidocaine need at least several injections of lidocaine to achieve even partial anesthesia. Such failures to become numb in the dental context have typically been explained away as being due either to the presence of an infection that lowers pH, or to the injection having “missed the nerve”.

Existing potassium therapies for various conditions include over-the-counter (OTC) potassium chloride and potassium gluconate. Potassium is bulky and not lipid soluble, so modern conveyances such as enteric coatings and transdermal patches are impractical for any significant doses. Liquid formulations suffer from bad taste or too much sugar and salt to mask that taste; salt and sugar also interfere with the body's ability to increase the serum potassium. Extended release formulations tend to use potassium chloride because it is the densest of the potassium salts. However, potassium chloride tastes awful and regularly causes gastric upset—resulting in very poor compliance, especially for a chronic condition. The potassium gluconate form is bulkier but better tolerated; more pills are needed for any given dose, but the higher number of pills creates a compliance challenge.

Problems in compliance with drug therapies have long been known (Kruse et al, 1994). Increased frequency of dosing within the day reduces compliance, with single dose drugs averaging 79% compliance and declined steadily to 51% in the case of a dose four times daily (Claxton et al, 2001), and these numbers decline further over time. In addition, compliance at night and on weekends is less reliable than in the morning (Kruse et al, 1994). Potassium salts typically last for a maximum of 6 hours, and extended release for 8-12 hours, so multiple doses each day are needed to maintain steady levels of higher serum potassium.

Individuals with this and other related conditions are distinguishable as a distinctive subtype by the partial or complete ineffectiveness of lidocaine as an anesthetic: these individuals have a “Lidocaine-Ineffective Condition”. Other key findings in SOS are inattention described as ADHD, painful muscle cramps, particularly in the extremities, and in females, severe PMS. These patients typically have their symptoms exacerbated by high sugar or salt meals.

I estimate that this condition affects 3% of the entire population, or ˜9 million people in the USA, which would make this syndrome among the most frequent causes of each of these diverse findings.

The DSM-5 criteria for ADHD are essentially inattention (and sometimes hyperactivity) that is non-syndromic, i.e., with no other significant findings (American Psychiatric Association 2013). Patients often describe the inattention as sensory overstimulation; a common metaphor is being in a room with many television sets, and being unable to control the sound to focus on just one. There are at least 203 syndromic diagnoses that have inattention as one of many findings, such as Lesch-Nyhan disease, Fragile X, and Tourette syndrome (Saul 2014; SimulConsult 2017). However, the DSM-5 diagnostic criteria for ADHD were written before widespread knowledge of the subtype of Sensory Overstimulation Syndrome (SOS), so those being evaluated for ADHD are not asked about potentially syndromic findings such as lidocaine ineffectiveness, cramps, and pain. Furthermore, the criteria for diagnosing ADHD requires symptomology of inattention before the age of 12. In women, these symptoms often first manifest after puberty (typically older than 12) as Premenstrual Syndrome (PMS) or cramps, pain and migraines during menstruation. Even if such findings are collected, today they are typically considered random co-morbidities, rather than part of the distinct syndrome of either ADHD or SOS.

The heritability of ADHD is ˜70-80% (Lesch et al. 2008, Franke et al. 2009), but no genetic cause has yet been identified, despite many studies. Although it has long been suspected that the abnormality in ADHD is in the brain and involves dopamine, the evidence for that assumption is weak, chiefly that dopamine-related drugs such as methylphenidate are effective in treating ADHD (DiMaio et al. 2003, Lasky-Su et al. 2008. Lesch et al. 2008, Franke et al. 2009, Gizer et al. 2009, Neale et al. 2010). Some mild associations with ADHD have been found for variants in genes DRD4 (dopamine receptor D4) and SLC6A3 (dopamine transporter) with odds ratios of 1.1-1.9 (DiMaio et al. 2003; Gizer et al. 2009). However, these modest effects may just reflect the known pharmacology that increasing dopamine improves mental focus in everyone, so these gene variants may just be penetrance factors for ADHD. Having a reliable test (i.e., Lidocaine effectiveness) to identify a homogeneous subgroup among those with ADHD will increase the chances of finding the first causative ADHD genes and providing appropriate treatments.

The pathogenesis of SOS is different from what has traditionally been believed about ADHD. Without wishing to be bound by theory, in the lidocaine-ineffective ADHD subgroup, I believe that the pathogenesis is NOT in the brain's dopamine system, but includes at minimum the sensory nerves. The sensory nerves of patients with SOS are too sensitive to stimuli, and produce excess signaling for the stimulus, resulting in the symptoms of overstimulation. Patients experience this as noises that sound louder than they do to others in the room without SOS, or lights that seem brighter, or clothes that are intolerably irritating, etc. Such overstimulation becomes overwhelming and often manifests as the deficit of attention (sometimes with hyperactivity) and is most often diagnosed as ADHD.

The level of potassium in the blood (“serum potassium”) regulates such signaling. Normal ranges of serum potassium in adults are 3.5-5.3 mEq/L. While serum potassium levels fluctuate within the normal range throughout the day in everyone, in these Lidocaine-Ineffective Conditions, some patients have episodes where their serum potassium falls below the normal level, described as hypokalemia, and to treat them and others, ways to increase their serum potassium are needed. Other patients remain within the normal range, however, when their serum potassium falls to the low end of the normal range they experience symptomatic episodes, even though such potassium levels produce no symptoms in controls.

It is important to emphasize that in a Lidocaine-Ineffective Condition, (e.g., SOS) serum potassium is neither low chronically nor outside the normal range of serum potassium levels most of the time. Nonetheless, Lidocaine-Ineffective Condition (e.g., SOS) patients benefit from increasing their level of serum potassium.

The condition is a chronic one, and patients benefit from increasing the serum potassium both prophylactically and acutely. I have discovered that patients with such “Lidocaine-Ineffective Conditions” that are treated with the compositions and methods of this invention (e.g., potassium supplementation or potassium-elevating agents) derive a significant therapeutic benefit. Clinical experience with 15 patients suggests that these patients can restore the normal level of nervous system sensitivity by potassium supplementation, which modestly increases levels of serum potassium and thus, suppresses the symptoms of ADHD by directly preventing sensory overstimulation.

There are parallels with the very rare muscle disease, Hypokalemic Periodic Paralysis (HypoPP), that affects ˜10 per million people or ˜3,000 people in the U.S., where paralysis episodes are the result of extreme episodes of overstimulation of muscle due to serum potassium falling below the normal level.

An important subset of HypoPP patients also have the findings of lidocaine insensitivity, ADHD and PMS. 80% of people with HypoPP patients have a known variant in either a sodium or calcium channel gene, resulting in HypoPP being part of a group of disorders of ion channels, referred to as channelopathies. In contrast, of the remaining ˜20% of people with HypoPP, there is a subgroup that manifest both HypoPP and lidocaine-insensitive ADHD, and are referred to as having the HypoPP+ (pronounced “HypoPP-plus”) form of HypoPP.

Even in HypoPP, potassium is neither low chronically nor outside the normal range of serum potassium levels most of the time. Instead, as in normal individuals, serum potassium fluctuates mostly within the normal range, e.g., dropping after a carbohydrate meal via an insulin-based mechanism. Nonetheless, a potassium level that would go unnoticed by normal people can trigger symptoms in people with HypoPP (Vicart et al. 2014, Segal et al. 2014).

HypoPP is often misdiagnosed as a psychiatric illness (“conversion disorder”), meaning the symptoms of paralysis are imagined. However, during paralysis episodes, the HypoPP patients have muscle swelling, and most patients have a known genetic variant that underlies the condition. Similarly, a Lidocaine-Ineffective Condition (e.g., SOS) symptoms have been treated as a psychological disorder. Without wishing to be limited by theory, I believe these symptoms are a manifestation of an unusually high need for certain minerals due to a problem with ion channels or transporters in tissues including the sensory nerves; and lacking that extra mineral level that experience extra signaling that proves to be distracting.

Diet modification in a Lidocaine-Ineffective Condition (e.g., SOS) is helpful but insufficient. For example, avoiding meals very high in sugar and salt, such as a classic teenage meal of a pizza and sugary soda, can help avoid acute episodes. However, diet modification is insufficient to treat a Lidocaine-Ineffective Condition (e.g., SOS), in part because many potassium rich foods, such as bananas and potatoes, come with high carbohydrates that trigger insulin release, which drives potassium from the serum into the cells. Such potassium-containing foods can even prove to be a net negative source of serum potassium.

Successful treatment of a Lidocaine-Ineffective Condition's (e.g., SOS) symptoms may include rigorous compliance with a prophylactic regimen that maintains higher levels of serum potassium. Apart from rare diseases such as HypoPP, high-dose potassium treatments are unusual outside of acute emergency interventions. Consequently, it had been thought that there was little need to find ways to deliver high doses of potassium as a tolerated, chronic therapy for large numbers of people, including children.

The present invention, includes novel ways to increase the serum potassium in such patients. The amount and frequency of potassium required daily and the requirement to continue the therapy over a patient's lifetime creates a need for better tolerated regimens with higher compliance. Potassium salts are inherently bulky and not lipid soluble, so they do not lend themselves to convenient delivery by methods such as patches and enteric coatings. A dense form of potassium salt, potassium chloride has an unpleasant taste and usually results in stomach upset and gastric pain. The better tolerated over-the-counter (OTC) potassium gluconate supplement pills weigh about 600 mg and are 1.5×0.5×0.5 cm in size. The typical adult dose needed to maintain adequately high serum potassium is 13.5 mEq taken four times in 24 hours, the equivalent of 24 OTC potassium tablets: a daunting regimen for any adult. The most common age of diagnosis of attention deficit is 7 years old, after children start school, and at that age, swallowing any pills can be problematic.

Certain drugs (such as renin-angiotensin-aldosterone system antagonists) that are designed to lower blood pressure can also increase blood potassium levels. However, the blood-pressure lowering effects of these drugs makes them poor monotherapies for patients with SOS who also have normal blood pressure, including most children and women with PMS.

In a Lidocaine-Ineffective Condition (e.g., SOS), night-time doses improve sleep quality which further helps to reduce symptomology. Since people with ADHD (and other Lidocaine-Ineffective Conditions) become particularly “compliance-challenged” when they are experiencing sensory overstimulation, creating an easy-to-follow, and high-compliance regimen improves outcomes.

I have devised treatments for Lidocaine-Ineffective Conditions, (which can also be used for Hypokalemic Conditions) that overcome problems with suboptimal treatment compliance and provides relief to these patients.

### Potassium Supplementation Agents

The first type of treatment involves treating a patient by administering potassium. For example, you can treat these conditions in a patient by administering to the patient a therapeutically effective amount of potassium or a potassium salt. The patient can be one who has been diagnosed as having a Lidocaine-Ineffective Condition, whether or not lidocaine effectiveness was tested, or a hypokalemic condition. In some embodiments, the method further includes diagnosing the patient as having a partial or complete lidocaine ineffectiveness prior to the administering the therapy. The method can include mineral treatments at adequate doses required to directly elevate serum potassium to levels that reduce or eliminate symptoms.

In some embodiments, the potassium salt can be potassium gluconate or chloride.

In some embodiments, the potassium is administered using a time-release (i.e., extended release) form to deliver a dose in a steady amount over a longer time.

Pharmaceutical compositions suitable for use in these methods can be provided in any suitable form, e.g., a (1) tablet (with or without coatings) or (2) capsule or in (3) liquid or (4) powder form or (5) a consumable food conveyance to accommodate the bulkiness of mineral salts and the required doses, especially for those unable to swallow pills. In addition, pharmaceutical compositions can be provided in ways that minimize significant carbohydrates and sugar to avoid the insulin effect that lowers serum potassium; and in ways that avoid surges in sodium consumption.

These ingredients may be prepared and packaged in ways that make compliance with the required regimen as easy and reliable as possible (for example, in kits).

The potassium dose over a 24-hours will vary by age and size of the patient. An adult will typically take 4-5 doses of 600 mg of elemental potassium, resulting in a dose over 24-hours of 2.4 g of elemental potassium, such as might be achieved with 13 g or 60 mEq or more of potassium gluconate; children will typically take doses of half that amount.

In some embodiments, the potassium is administered to the patient in multiple doses, e.g., from 1 to 30 dosage forms (e.g., pills or a food conveyance such as a potassium-enriched nutritional bars), such as such as in 1 to 24, 2 to 20, 2 to 15, 2 to 12, 2 to 9, 3 to 8, 2 to 7, or 3 to 6 dosage forms (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 dosage forms). A total of dose may be administered to a patient per 24 hours, for example, in the range of approximately 90 mg to 5,000 mg of elemental potassium, e.g., 90 to 1000 mg, 250 to 4000 mg, 500 to 4000 mg, 750 to 4000 mg, 1000 to 4000 mg, 1250 to 4000, 1500 to 4000 mg. 2000 to 4000 mg, 1000 to 2000 mg, 1000 to 3000, or 3000 to 5000 mg. Such daily doses could be about: 90 mg, 100 mg, 125 mg, 150 mg, 175 mg, 200 mg, 225 mg, 250 mg, 500 mg, 750 mg, 1,000 mg, 1,050 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg, 3,000 mg, 3,500 mg, 4,000 mg, 4,500 mg, or 5,000 mg of elemental potassium per day. For example, a child may be administered from about 250 mg per dose of elemental potassium, and an adult may be administered from about 500 mg per dose of elemental potassium, and each may take 4-5 such doses in 24 hours.

### Potassium-Elevating Agents

In another aspect, the method can involve treating a human patient by administering to the patient a therapeutically effective amount of a potassium-elevating agent, such as a renin-angiotensin-aldosterone system antagonist that elevates serum potassium. The patient can be one who has been diagnosed as having a Lidocaine-Ineffective Condition, whether or not lidocaine effectiveness was tested, or hypokalemic condition. In some embodiments, the method further includes diagnosing the patient as having a partial or complete lidocaine ineffectiveness prior to the administering.

The methods include elevating serum potassium indirectly (e.g., through renin-angiotensin-aldosterone system antagonists) to levels that reduce or eliminate symptoms. The methods may further include administering blood pressure boosting drugs (e.g., by a mechanism other than the renin-angiotensin-aldosterone system such as fludrocortisone or midodrine), to allow those (e.g., children) with normal or low blood pressure to be treated safely.

A variety of renin-angiotensin-aldosterone system antagonists and other potassium-elevating drugs may be used in the compositions and methods described herein. Renin is a protease that converts angiotensin to angiotensin I, which is in turn cleaved by angiotensin-converting enzyme (ACE) to form angiotensin II, which acts on the adrenal cortex to induce the release of aldosterone and the subsequent excretion of aqueous potassium. An inhibitor of one or more of the components of the renin-angiotensin-aldosterone system results in elevated retention of potassium. Renin-angiotensin-aldosterone system antagonists include compounds capable of inhibiting one or more components of the renin-angiotensin system cascade, thereby attenuating the excretion of potassium and elevating serum potassium concentration. Exemplary renin-angiotensin-aldosterone system antagonists include renin inhibitors, ACE inhibitors, angiotensin receptor antagonists, and aldosterone antagonists, such as those described herein and known in the art.

Exemplary renin inhibitors that may be used in conjunction with the compositions and methods described herein include aliskiren and compounds structurally related thereto, such as those described, for example, in U.S. Pat. No. 5,719,141 and International Patent Application No. WO 2001/009079, the disclosures of each of which are incorporated herein by reference, as they pertain to renin inhibitors. Exemplary renin inhibitors useful in conjunction with the compositions and methods described herein additionally include enalkiren and compounds structurally related thereto, remikiren and compounds structurally related thereto, among other renin inhibitors, such as those described in U.S. Pat. Nos. 4,814,342; 4,855,303; 4,895,834; and 5,696,116, the disclosures of each of which are incorporated herein by reference as they pertain to renin inhibitors.

Exemplary ACE inhibitors that may be used in conjunction with the compositions and methods described herein include benazepril and its metabolite benazeprilat and compounds structurally related thereto, such as those described in U.S. Pat. No. 4,410,520, the disclosure of which is incorporated herein by reference as it pertains to ACE inhibitors. Additional examples of ACE inhibitors that may be used in conjunction with the compositions and methods described herein include captopril and compounds structurally related thereto, such as those described in US Patent No. U.S. Pat. No. 4,105,776, the disclosure of which is incorporated herein by reference as it pertains to ACE inhibitors. ACE inhibitors useful in conjunction with the compositions and methods described herein additionally include enalapril, lisinopril and compounds related structurally thereto, such as those described in U.S. Pat. Nos. 4,374,829; 6,468,976; and 6,465,615, the disclosures of each of which are incorporated herein by reference as they pertain to ACE inhibitors. Exemplary ACE inhibitors useful in conjunction with the compositions and methods described herein additionally include perindopril erbumine, the ethyl ester thereof, and compounds related thereto, as well as quinapril and compounds related thereto, ramipril, the ethyl ester thereof, and compounds related thereto, fosinopril sodium salt and compounds related thereto, moexipril and compounds related thereto; and imidapril and compounds related thereto, among other ACE inhibitors, such as those described in U.S. Pat. Nos. 5,696,116; 6,410,524; and 6,482,797, the disclosures of each of which are incorporated herein by reference as they pertain to ACE inhibitors.

Angiotensin receptor antagonists that may be used in conjunction with the compositions and methods described herein include, without limitation, losartan and various substituted imidazole derivatives and other compounds related thereto, such as those described in U.S. Pat. No. 5,138,069; the disclosure of which is incorporated herein by reference as it pertains to angiotensin receptor antagonists. Additional examples of angiotensin receptor antagonists include valsartan and compounds related thereto, such as those described in U.S. Pat. No. 5,399,578, the disclosure of which is incorporated herein by reference as it pertains to angiotensin receptor antagonists. Exemplary angiotensin receptor antagonists additionally include irbesartan and compounds related thereto, such as those described in U.S. Pat. Nos. 5,270,317 and 5,352,788, the disclosure of which is incorporated herein by reference as it pertains to angiotensin receptor antagonists. Additional angiotensin receptor antagonists include candesartan and compounds related thereto, such as those described in U.S. Pat. No. 5,196,444, the disclosure of which is incorporated herein by reference as it pertains to angiotensin receptor antagonists. Exemplary angiotensin receptor antagonists also include telmisartan and compounds related thereto, tasosartan and compounds related thereto, such as those described in U.S. Pat. No. 5,149,699, the disclosure of which is incorporated herein by reference as it pertains to angiotensin receptor antagonists. Exemplary angiotensin receptor antagonists additionally include eprosartan and compounds related thereto, such as those described in U.S. Pat. No. 5,185,351, the disclosure of which is incorporated herein by reference as it pertains to angiotensin receptor antagonists. Angiotensin receptor antagonists additionally include saralasin, an octapeptide analog of bovine angiotensin II in which amino acid residues 1 and 8 are substituted with sarcosine and alanine, respectively. Additional examples of angiotensin receptor antagonists that may be used in conjunction with the compositions and methods described herein include those described in U.S. Pat. Nos. 5,484,780; 6,028,091; and 6,329,384, the disclosures of each of which are incorporated herein by reference as they pertain to angiotensin receptor antagonists.

Exemplary aldosterone antagonists useful in conjunction with the compositions and methods described herein include, without limitation, eplerenone and compounds related thereto, such as those described in U.S. Pat. No. 4,559,332, the disclosure of which is incorporated herein by reference as it pertains to aldosterone antagonists. Aldosterone antagonists additionally include spironolactone alone or in combination with hydrochlorothiazide, and compounds related thereto. Exemplary aldosterone antagonists useful in conjunction with the compositions and methods described herein additionally include those described in U.S. Pat. No. 6,410,524, the disclosure of which is incorporated herein by reference.

In certain embodiments, the potassium-elevating agent may be formulated in a pill, tablet, capsule, powder, liquid, or food conveyance. The potassium-elevating agent, e.g., renin-angiotensin-aldosterone system antagonist, may be administered at a dose of from 0.05 mg/day to 600 mg/day, e.g., 0.05 to 50, 10 to 100, 10 to 200, 10 to 300, 100 to 500, 100 to 400, 100 to 300, 200 to 600, or 300 to 600 mg/day (e.g., about 10 mg/day, 20 mg/day, 30 mg/day, 40 mg/day, 50 mg/day, 60 mg/day, 70 mg/day, 80 mg/day, 90 mg/day, 100 mg/day, 200 mg/day, 300 mg/day, 400 mg/day, 500 mg/day, or 600 mg/day). The potassium-elevating agent, e.g., renin-angiotensin-aldosterone system antagonist, may be administered to the patient in 1 to 30 dosage forms, such as in 1 to 24, 2 to 20, 2 to 15, 2 to 12, 2 to 9, 3 to 8, 2 to 7, or 3 to 6 dosage forms (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30). The potassium-elevating agent may be packaged in a kit. The potassium-elevating agent may be formulated for extended release.

### Additional Therapeutic Agents

Potassium or potassium-elevating agents may be administered in combination with one or more additional therapeutic agents.

Attention deficit and other Lidocaine-Ineffective Conditions have other treatments and are known to often occur with other co-morbidities, including depression, anxiety, insomnia and pain. Accordingly, it is an aspect of the present invention that patients treated for low serum potassium by one of the above methods can often also treated with other therapeutic agents, which may also be used for patients with a Lidocaine-Ineffective Condition, e.g., SOS.

In some embodiments, the additional agent is a TAAR1 agonist, an inhibitor of neurotransmitter reuptake of one or more of norepinephrine, dopamine, and serotonin, an alpha-2 adrenergic receptor agonist, a monoamine oxidase inhibitor, an adenosine receptor antagonist, various herbal or other natural ingredients; a barbiturate; a benzodiazepine; a hypnotic agent; an antihistamine; pyrazolopyrimidine; a serotonin antagonist and reuptake inhibitor (SARI) a selective serotonin reuptake inhibitor (SSRI); a beta blocker; a serotonin-norepinephrine reuptake inhibitor (SNRI); a tricyclic antidepressant (TCA); a tetracyclic antidepressant; an antipsychotic; an opioid; a folate treatment; a treatment for mania; a serotonin modulator and stimulator (SMS); a vitamin B3 complex component; a treatment for hypothyroidism; a muscle relaxant; an anticonvulsant; NSAID; and/or a diuretic and/or ingredients that are used to minimize the stomach upset that can accompany large doses of potassium.

Examples of suitable TAAR1 agonists include amphetamine, levoamphetamine, dextroamphetamine, and lisdexamfetamine. Examples of inhibitors of reuptake include methylphenidate, dexmethylphenidate, atomexetine, modafinil, armodafinil, bupropion, and venlafaxine. Examples of alpha-2 adrenergic receptor agonists include clonidine and guanfacine. Examples of monoamine oxidase inhibitors include selegiline, tranylcypromine, and phenelzine. Examples of adenosine receptor antagonists include caffeine, theophylline, and theobromine. Examples of herbal or other natural ingredients include acontium napellus, chocolate, cinchona officinalis, coffee, gnaphalium polycephalum, guarana, guayusa, leduum palustre, magnesia phosphorica, rhus toxicodendron, tea, viscum album, Hypericum, yaupon, and khat. Examples of NSAIDs include aspirin, diclofenac, diflunisal, indomethacin, sulindac, etodolac, mefenamic acid, meclofenamate, flufenamic acid, tolmetin, ketorolac, diclofenac, ibuprofen, naproxen, fenoprofen, ketoprofen, flurbiprofen, oxaprozin, piroxicam, meloxicam, nabumetone, celecoxib, valdecoxib, parecoxib, etoricoxib, and lumaricoxib. Examples of barbiturate is secobarbital, pentobarbital, phenobarbital, amobarbital, or butabarbital. Examples of benzodiazepine is alprazolam, diazepam, lorazepam, temazepam, clonazepam, oxazepam, quazepam, flurazepam, adinazolam, estazolam, flubromazolam, nitrazolam, pyrazolam, triazolam, or zapizolam. Examples of hypnotic agent is chloral hydrate, eszopiclone, tasimelteon, zolpidem, ramelteon, SAR, melatonin, agomelatine, tasimelteon, TIK-301, or suvorexant. Examples of antihistamine is acrivastine, azelastine, acrivastine, cetirizine, diphenhydramine, bilastine, bromodiphenhydramine, brompheniramine, buclizine, carbinoxamine, chlorodiphenhydramine, chlorphenamine, chlorpromazine, clemastine, cyclizine, cyproheptadine, dexbrompheniramine, dexchlorpheniramine, dimenhydrinate, dimetindene, doxylamine, ebastine, embramine, fexofenadine, loratidine, hydroxyzine, meclizine, mirtazapine, olopatadine, orphenadrine, phenindamine, pheniramine, phenyltoloxamine, promethazine, rupatadine, tripelennamine, or triprolidine. In some embodiments, an antihistamine may be administered in combination with a nonsteroidal anti-inflammatory drug (NSAID). Examples of pyrazolopyrimidines include zaleplon, indiplon, ocinaplon, divaplon, or lorediplon. Examples of SARIs include trazodone, nefazodone, mepiprazole, lubazodone, loriprazole, or etoperidone. Examples of SSRIs include sertraline, escitalopram, fluoxetine, citalopram, or paroxetine. Examples of beta blockers include propranolol or atenolol. Examples of SNRIs include duloxetine, venlafaxine, desvenlafaxine, atomozetine, milnacipran, or levomilnacipran. Examples of TCAs include nortriptyline, imipramine, amoxapine, desipramine, dibenzocycloheptadiene, trimipramine, doxepin, amitriptyline/chlordiazepoxide, clomipramine, amitriptyline/perphenazine, or protriptyline. Examples of tetracyclic antidepressants include mirtazapine, maprotiline, or a piperazino-azepine. Examples of antipsychotics include aripiprazole, olanzapine, risperidone, paliperidone, or brexipiprazole. Examples of opioids include codeine, morphine, thebaine, oripavine, diacetylmorphine, nicomorphine, dipropanoylmorphine, diacetyldihydromorphine, acetylpropionylmorphine, desomorphine, methyldesorphine, dibenzoylmorphine, dihydrocodeine, ethylmorphine, heterocodeine, buprenorphine, etorphine, hydrocodone, hydromorphone, oxycodone, oxymorphone, fentanyl, alphamethylfentanyl, alfentanil, sufentanil, remifentanil, carfentanyl, ohmefentanyl, pethidine, ketobemidone, mppp, allylprodine, prodine, pepap, promedol, propoxyphene, dextropropoxyphene, dextromoramide, bezitramide, piritramide, methadone, dipipanone, levomethadyl acetate, difenoxin, diphenoxylate, loperamide, dezocine, pentazocine, phenazocine, buprenorphine, dihydroetorphine, etorphine, butorphanol, nalbuphine, levorphanol, levomethorphan, racemethorphan, lefetamine, menthol, meptazinol, mitragynine, tilidine, tramadol, tapentadol, eluxadoline, AP-237, and 7-hydroxymitragynine. Examples of folate treatments include vitamin B12 and folic acid. Examples of treatments for mania include lithium, quetiapine, and valproate. Examples of SMSs include vilazodone and vortioxetine. Examples of vitamin B3 complex components include nicotinic acid (niacin) and nicotinamide (niacinamide). An example of the treatment for hypothyroidism is desiccated thyroid. Examples of muscle relaxants include cyclobenzaprine or tizanidine. Examples of anticonvulsants include lamotrigine, pregabalin, or gabapentin. Diuretics include (a) a thiazide-based diuretic; (b) a loop-based diuretic; (c) a potassium-sparing diuretic; (d) pamabrom; and (e) mannitol. Examples of thiazide-based diuretics include indapamide, hydrochlorothiazide, chlorthalidone, chlorothiazide, metolazone, methyclothiazide, bendroflumethiazide, polythiazide, or hydroflumethiazide. Examples of loop-based diuretics include bumetanide, thacrynic acid, torsemide, or ethacrynic acid. Examples of potassium-sparing diuretics include triamterene; spironolactone, or amiloride. In some embodiments, a potassium-sparing diuretic is administered with a thiazide (e.g., hydrochlorothiazide). Ingredients that are used to minimize the stomach upset that can accompany large doses of potassium include bismuth subsalicylate, calcium carbonate, and ranitidine.

Additional therapeutic agents further include carbamazepine, pemoline, buspirone, acetaminophen, and metadoxine.

As is known in the art, certain of these agents are also known to be stimulants (e.g., amphetamine, methylphenidate, modafinil, caffeine, and similar agents).

### Pharmaceutical Compositions, Kits, and Routes of Administration

The invention also features pharmaceutical compositions and pharmaceutical compositions formulated in kits.

Administration of the compounds described herein may be by any suitable means that results in treatment. The therapeutic agents described herein may be contained in an appropriate amount in one or more suitable carrier substances and may be present in amounts totaling 1-95% by weight of the total weight of the composition. If appropriate, the composition may be provided in a dosage form that is suitable for oral, parenteral (e.g., intramuscular), rectal, cutaneous, subcutaneous, subdermal (with or without a customized dose delivery pattern), topical, transdermal (e.g., patch, patch pump), transmucosal, buccal, sublingual, nasal, vaginal, epidural, otic, or ocular administration, or by injection (e.g., subcutaneous, intramuscular, and intravenous), inhalation, or direct contact with the nasal or oral mucosa (such as sublingual or buccal). In certain embodiments, the dosage is formulated for extended release, e.g., over a period of 4, 6, 8, 10, 12, 16, 18, 20, 22, or 24 hours.

The pharmaceutical compositions and routes of administration may be in the form of, for example, tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols.

In some embodiments, the composition may be in the form of a food conveyance, e.g., food (e.g., medical food) or beverage formulation, that accommodates the bulk of potassium salts (e.g., treatments mixed into a nutrition bar with appropriately low sugar and salt in order to avoid an insulin effect that lowers serum potassium; and in ways that avoid surges in sodium consumption).

These ingredients may be prepared and packaged in ways that make compliance with the required regimen as easy and reliable as possible (for example, in kits). In the kits of the invention, the active agents may or may not be formulated together, when multiple agents are present. The potassium in the kit may be formulated in a food conveyance or for extended release.

When formulated together for at least one dose, the kit will include multiple dosages. In one embodiment, when an additional therapeutic agent is present (e.g., a stimulant or a blood-pressure raising medicine), the kit includes fewer doses of the additional agent relative to the potassium-elevating agent, e.g., renin-angiotensin-aldosterone system antagonist, or relative to potassium. For example, the kit may include paired doses of another therapeutic agent and the potassium-elevating agent, e.g., renin-angiotensin-aldosterone system antagonist, or potassium, except for one additional dose of the potassium-elevating agent, e.g., renin-angiotensin-aldosterone system antagonist, or potassium intended to be taken before bed time. Alternatively, the kit may include multiple doses of the combined agents with an additional dose, intended for administration prior to bed time, including the potassium-elevating agent, e.g., renin-angiotensin-aldosterone system antagonist, or potassium but not the additional agent. The dose not including the additional agent may have a different appearance, shape, or form to distinguish.

The compositions may be formulated according to conventional pharmaceutical practice (see, e.g., Remington: The Science and Practice of Pharmacy (2012, 22^{nd} ed.) and in The United States Pharmacopeia: The National Formulary (2015, USP 38 NF 33).

Each compound may be formulated in a variety of ways that are known in the art. For example, the therapeutic agents described herein can be formulated together or separately. The individually or separately formulated agents can be packaged together as a kit. Non-limiting examples include but are not limited to kits that contain, e.g., a food conveyance and a pill, two pills, a pill and a powder, a suppository and a liquid in a vial, two topical creams, and the like. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions.

The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like. Kits may be packaged for use in a single day, e.g., two to six doses to be taken in a single day, for a week, e.g., one to six doses to be taken for seven days, for a school/work week, e.g., one to six doses to be taken for five days, or a weekend, e.g., one to six doses to be taken for three days.

Formulations for oral use include tablets containing the active ingredient(s) in a mixture with non-toxic pharmaceutically acceptable excipients. These excipients may be, for example, inert diluents or fillers (e.g., mannitol or microcrystalline cellulose); granulating and disintegrating agents (e.g., cellulose derivatives including microcrystalline cellulose, croscarmellose, alginates, or alginic acid); binding agents (e.g., acacia, alginic acid, sodium alginate, gelatin, microcrystalline cellulose, magnesium aluminum silicate, carboxymethylcellulose, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., microcrystalline cellulose or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution- or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions described herein can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

The solid forms of consumable food can be made of the standard ingredients of consumable food, either raw or baked, such as dried fruits, nuts, and various grains. Importantly, the level of salts and carbohydrates need to be carefully considered to avoid reducing the benefit of the potassium.

Compositions suitable for topical application, if appropriate, can be formulated as a useful topical composition, e.g., a cream, an ointment, a paste, a lotion, a gel, a solution, a suspension, a spray, a foam, a patch, or a tincture. Topical compositions may be administered dermally or transdermally. Typical topical compositions are formulated in a pharmaceutically acceptable vehicle suitable for topical application to the skin. Examples of such vehicles include water, alcohol, or an oil, or a mixture thereof. Further excipients that may be used in the topical compositions include colorants, dyestuffs, fragrances, deodorants, thickeners, antioxidants, solvents, surfactants, detergents, gelling agents, fillers, viscosity-controlling agents, preservatives, humectants, moisturizers, emollients, hydration agents, chelating agents, tonicity adjusting agents, solubilizing excipients, dispersants, permeation enhancer agents, plasticizing agents, preservatives, stabilizers, demulsifiers, wetting agents, sunscreens, emulsifiers, and astringents.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve, which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer.

Compositions suitable for buccal or sublingual administration include tablets, lozenges, and pastilles, where the active ingredient is formulated with a carrier, such as sugar, acacia, tragacanth, or gelatin and glycerine. Compositions for rectal or vaginal administration are convenient in the form of suppositories containing a conventional suppository base, such as cocoa butter.

Generally, when administered to a human, the dosage of any of the compounds, alone or in combination, will depend on the nature of the compound, and can readily be determined by one skilled in the art and described herein.

Administration of each drug in a combination therapy, as described herein, can, independently, be one or more times daily for, e.g., from one day to one year or more (e.g., at least 1 week, 2 weeks, 3 weeks, 4 weeks, 1 month, 2 months, 3 months, 4 months, 5 months, 6 months, 7 months, 8 months, 9 months, 10 months, 11 months, 12 months), and may even be for the life of the patient.

In some embodiments, the formulation includes various approaches to improve bioavailability, e.g., gut absorption, including (but not limited to) crystalline solid formulations, amorphous formulations, lipid formulations, and self-emulsifying systems.

### Methods of Treatment

The compositions described herein can be used to treat a Lidocaine-Ineffective Condition or hypokalemic condition. The present invention administers either a potassium-elevating agent, e.g., a renin-angiotensin-aldosterone system antagonist, or potassium, with or without additional agents, for the treatment of these conditions. When multiple agents are employed, administration may occur in the same or different dosage forms. When two or more agents are employed, they also may or may not be administered at the same time point. For example, the agents may be administered within 6 hours, e.g., within 3, 2, 1, 0.5, or 0.25 hours of each other. When the therapeutic is a stimulant (e.g., amphetamine, methylphenidate, modafinil, caffeine, or similar agent), preferably, the methods include administering the stimulant to take effect primarily during waking hours. For example, any doses taken within 6 hours, e.g., within 3, 2, or 1 hours of bedtime may not include a stimulant to allow the patient to sleep normally. Preferably, the amount of therapeutic typically administered to treat a Lidocaine-Ineffective Condition or hypokalemic condition is reduced when the renin-angiotensin-aldosterone system antagonist or potassium is co-administered; for example, the amount of another therapeutic agent is reduced by at least 25, 50, 75, 80, 85, 90, or 95%.

To assess whether a patient has a Lidocaine-Ineffective Condition, one of skill in the art can determine whether the patient is sensitive to lidocaine anesthesia, e.g., as described in PCT Publication No. WO2017/035470. Diagnosis of a hypokalemic condition, disorder of attention, Asperger Syndrome, Sensory Overstimulation Syndrome (SOS), Sensory Processing Disorder, Fibromyalgia, various other pain syndromes and/or Premenstrual Syndrome can be carried out using known methods.

### EXAMPLES

Based on our research and discussions with us, patients with lidocaine-ineffective conditions have been treated with potassium and potassium-elevating drugs under the care of their personal physician in off-label use. Those patients report the approach is “nothing short of miraculous” (middle-aged lawyer), that their child “stopped fighting with his teacher” (mother of 10-year-old boy), and that the treatment “continued to be as effective, at the same dose, after many months” (middle-aged woman).

Efficacy of the use of potassium in treating a Lidocaine-Ineffective Condition is also assessed in a placebo controlled trial. The trial has 2 arms, lidocaine sensitive (effective) and lidocaine insensitive (ineffective). Within each arm, subjects are randomized on a 1-to-1 allocation to the treatment or control groups. Lidocaine effectiveness is assessed using a non-invasive, pain-free testing using taste following application of lidocaine to the tongue. The effectiveness of potassium supplementation in an ADHD population with lidocaine taste insensitivity is assessed and compared with that for ADHD subjects for whom lidocaine is effective, using a randomized study design with ADHD-RS-V, Quotient (http://www.quotient-adhd.com/; Infante et al. 2015, Teicher et al. 2012)) and CGI testing.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

### REFERENCES

Burcu M, Zito J, Metcalfe L et al (2016)
   Trends in Stimulant Medication Use in Commercially Insured Youths and Adults, 2010-2014, JAMA Psychiatry 73(9):992-993
Claxton, A J, Cramer, J, Pierce C (2001) A systematic review of the associations between dose regimens and medication compliance. Clinical Therapeutics, 23(8):1296-1310
Dalsgaard S, Østergaard S D, Leckman J F et al. (2015) Mortality in children, adolescents, and adults with attention deficit hyperactivity disorder: a nationwide cohort study. Lancet. 385:2190-6.
DiMaio S, Grizenko N, Joober R (2003) Dopamine genes and attention-deficit hyperactivity disorder: a review. J Psychiatry Neurosci. 28:27-38.
Franke B, Neale B M, Faraone S V (2009) Genome-wide association studies in ADHD. Hum Genet. 126:13-50.
Gizer I R, Ficks C, Waldman I D (2009) Candidate gene studies of ADHD: a meta-analytic review. Hum Genet. 126:51-90.
Kruse W, Rampmaier J, Ullrich G, Weber E (1994) International Journal of Clinical Pharmacology and Therapeutics 32(9):452-457
Infante M A, Moore E M, Nguyen T T, Fourligas N, Mattson S N, Riley E P (2015) Objective assessment of ADHD core symptoms in children with heavy prenatal alcohol exposure. Physiol Behav. 148:45-50.
Lasky-Su J1, Neale B M, Franke B, et al. (2008) Genome-wide association scan of quantitative traits for attention deficit hyperactivity disorder identifies novel associations and confirms candidate gene associations. Am J Med Genet B Neuropsychiatr Genet. 2008 5; 147B:1345-54.
Lesch K P, Timmesfeld N, Renner T J et al. (2008) Molecular genetics of adult ADHD: converging evidence from genome-wide association and extended pedigree linkage studies. J Neural Transm (Vienna). 115:1573-85
Levitt J O (2008) Practical aspects in the management of hypokalemic periodic paralysis. J Translational Medicine 6:18
Nakai Y, Milgrom P, Mancl L et al. (2000) Effectiveness of local anesthesia in pediatric dental practice. J Am Dent Assoc. 131:1699-705.
Neale B M, Medland S E, Ripke S et al. (2010) Meta-analysis of genome-wide association studies of attention-deficit/hyperactivity disorder. J Am Acad Child Adolesc Psychiatry. 49:884-97.
Rozanski R J, Primosch R E, Courts F J (1988). Clinical efficacy of 1 and 2% solutions of lidocaine. Pediatr Dent. 10:287-90.
Saul R (2014) “ADHD Does Not Exist”. HarperCollins.
Segal M M (2014). We Cannot Say Whether Attention Deficit Hyperactivity Disorder Exists, but We Can Find Its Molecular Mechanisms. Pediatric Neurology; 51:15-16
Segal M M (2015) Devices, Kits, and Methods for Determining Sensitivity to Anesthetics. US Patent Filing 62/210,747, Filed Sep. 14, 2015
Segal M M (2016) Therapies for the Treatment of Lidocaine-Ineffective Conditions. US Patent Filing 62/425,512, Filed Dec. 2, 2016
Segal M M, Jurkat-Rott K, Levitt J, Lehmann-Horn F (2014) Hypokalemic periodic paralysis—an owner's manual. http://simulconsult.com/resources/hypopp.html
Segal M M, Rogers G F, Needleman H L, Chapman C A (2007) Hypokalemic sensory overstimulation. J Child Neurol. 22:1408-1410
SimulConsult Diagnostic Decision Support 2017 (www.simulconsult.com)
Teicher M H, Polcari A, Fourligas N, Vitaliano G, Navalta C P (2012) Hyperactivity persists in male and female adults with ADHD and remains a highly discriminative feature of the disorder: a case-control study. BMC Psychiatry. 12:190.
Vicart S, Sternberg S, Arzel-Hézode M et al. (2014) Hypokalemic Periodic Paralysis. GeneReviews. http://www.ncbi.nlm.nih.gov/books/NBK1338/ accessed 31 Aug. 2017.

What is claimed is:

1. A method of treating a Lidocaine-Ineffective Condition in a human patient, the method comprising administering to the patient a therapeutically effective amount of potassium formulated for extended release, in a food conveyance, or in a kit or a therapeutically effective amount of potassium and one or more additional therapeutic agents.

2. A method of treating a Lidocaine-Ineffective Condition in a human patient, the method comprising administering to the patient a therapeutically effective amount of a potassium-elevating agent.

3. A method of treating a Hypokalemic Condition in a human patient, the method comprising administering to the patient a therapeutically effective amount of potassium formulated in a food conveyance or in a kit or a therapeutically effective amount of potassium and one or more additional therapeutic agents.

4. A method of treating a Hypokalemic Condition in a human patient, the method comprising administering to the patient a potassium-elevating agent formulated in a food conveyance or in a kit or a therapeutically effective amount of a potassium-elevating agent and one or more additional therapeutic agents.

5. The method of claim 1 or 2, wherein prior to administering the treatment the patient is diagnosed as having partial or complete ineffectiveness of the anesthetic lidocaine.

6. The method of claim 1 or 3, wherein the potassium comprises potassium gluconate or potassium chloride.

7. The method of claim 1 or 2, wherein the administering is oral, subdermal, ocular, otic, vaginal, rectal, IV, intranasal, or transdermal.

8. The method of claim 1 or 3, wherein the potassium is formulated in a pill, tablet, capsule, powder, liquid, or food conveyance.

9. The method of claim 1 or 3, wherein 90 mg to 5,000 mg of elemental potassium is administered.

10. The method of claim 1 or 3, wherein the potassium is administered to the patient in 1 to 30 dosage forms.

11. The method of claim 1 or 3, wherein the potassium is packaged in a kit.

12. The method of claim 1 or 3, wherein the potassium is formulated for extended release.

13. The method of claim 2 or 4, wherein the potassium-elevating agent is formulated for extended-release.

14. The method of claim 1 or 2, wherein the Lidocaine-Ineffective Condition is Sensory Overstimulation Syndrome.

15. The method of claim 2, further comprising administering an additional therapeutic agent.

16. The method of claim 1 or 15, wherein the additional therapeutic agent is a TAAR1 agonist, an inhibitor of neurotransmitter reuptake of one or more of norepinephrine, dopamine, and serotonin, an alpha-2 adrenergic receptor agonist, a monoamine oxidase inhibitor, an adenosine receptor antagonist, a barbiturate; a benzodiazepine; a hypnotic agent; an antihistamine, a pyrazolopyrimidine; a serotonin antagonist and reuptake inhibitor (SARI); a selective serotonin reuptake inhibitor (SSRI); a beta blocker; a serotonin-norepinephrine reuptake inhibitor (SNRI); a tricyclic antidepressant (TCA); a tetracyclic antidepressant; an antipsychotic; an opioid; a folate treatment; a treatment for mania; a serotonin modulator and stimulator (SMS); a vitamin B3 complex component; a treatment for hypothyroidism; a muscle relaxant; an anticonvulsant; a diuretic; or a stomach medicine.

17. The method of claim 16, wherein the TAAR1 agonist is selected from the group consisting of amphetamine, levoamphetamine, dextroamphetamine, and lisdexamfetamine; and/or the inhibitor of reuptake is selected from the group consisting of methylphenidate, dexmethylphenidate, atomexetine, modafinil, armodafinil, bupropion, and venlafaxine; and/or
wherein the alpha-2 adrenergic receptor agonist is selected from the group consisting of clonidine and guanfacine; and/or
wherein the monoamine oxidase inhibitor is selegiline, tranylcypromine, or phenelzine; and/or
wherein the additional therapeutic agent is selected from the group consisting of carbamazepine, pemoline, buspirone, acetaminophen, and metadoxine; and/or
wherein the adenosine receptor antagonist is selected from the group consisting of caffeine, theophylline, and theobromine; and/or
wherein the additional therapeutic agent is selected from the group consisting of acontium napellus, chocolate, cinchona officinalis, coffee, gnaphalium polycephalum, guarana, guayusa, leduum palustre, magnesia phosphorica, rhus toxicodendron, tea, viscum album, Hypericum, yaupon, and khat; and/or
wherein the additional therapeutic agent is a nonsteroidal anti-inflammatory drug (NSAID); and/or
wherein the NSAID is aspirin, diclofenac, diflunisal, indomethacin, sulindac, etodolac, mefenamic acid, meclofenamate, flufenamic acid, tolmetin, ketorolac, diclofenac, ibuprofen, naproxen, fenoprofen, ketoprofen, flurbiprofen, oxaprozin, piroxicam, meloxicam, nabumetone, celecoxib, valdecoxib, parecoxib, etoricoxib, or lumaricoxib; and/or
wherein the barbiturate is secobarbital, pentobarbital, phenobarbital, amobarbital, or butabarbital; and/or
wherein the benzodiazepine is alprazolam, diazepam, lorazepam, temazepam, clonazepam, oxazepam, quazepam, flurazepam, adinazolam, estazolam, flubromazolam, nitrazolam, pyrazolam, triazolam, or zapizolam; and/or
wherein the hypnotic agent is chloral hydrate, eszopiclone, tasimelteon, zolpidem, ramelteon, SAR, melatonin, agomelatine, tasimelteon, TIK-301, or suvorexant; and/or
wherein the antihistamine is acrivastine, azelastine, acrivastine, cetirizine, diphenhydramine, bilastine, bromodiphenhydramine, brompheniramine, buclizine, carbinoxamine, chlorodiphenhydramine, chlorphenamine, chlorpromazine, clemastine, cyclizine, cyproheptadine, dexbrompheniramine, dexchlorpheniramine, dimenhydrinate, dimetindene, doxylamine, ebastine, embramine, fexofenadine, loratidine, hydroxyzine, meclizine, mirtazapine, olopatadine, orphenadrine, phenindamine, pheniramine, phenyltoloxamine, promethazine, rupatadine, tripelennamine, or triprolidine; and/or
wherein the pyrazolopyrimidine is zaleplon, indiplon, ocinaplon, divaplon, or lorediplon; and/or
wherein the SARI is trazodone, nefazodone, mepiprazole, lubazodone, loriprazole, or etoperidone; and/or
wherein the SSRI is sertraline, escitalopram, fluoxetine, citalopram, or paroxetine; and/or
wherein the beta blocker is propranolol or atenolol; and/or
wherein the SNRI is duloxetine, venlafaxine, desvenlafaxine, atomozetine, milnacipran, or levomilnacipran; and/or
wherein the TCA is nortriptyline, imipramine, amoxapine, desipramine, dibenzocycloheptadiene, trimipramine, doxepin, amitriptyline/chlordiazepoxide, clomipramine, amitriptyline/perphenazine, or protriptyline; and/or
wherein the tetracyclic antidepressant is mirtazapine, maprotiline, or a piperazino-azepine; or
wherein the antipsychotic is aripiprazole, olanzapine, risperidone, paliperidone, or brexipiprazole; and/or
wherein the opioid is codeine, morphine, thebaine, oripavine, diacetylmorphine, nicomorphine, dipropanoylmorphine, diacetyldihydromorphine, acetylpropionylmorphine, desomorphine, methyldesorphine, dibenzoylmorphine, dihydrocodeine, ethylmorphine, heterocodeine, buprenorphine, etorphine, hydrocodone, hydromorphone, oxycodone, oxymorphone, fentanyl, alphamethylfentanyl, alfentanil, sufentanil, remifentanil, carfentanyl, ohmefentanyl, pethidine, ketobemidone, mppp, allylprodine, prodine, pepap, promedol, propoxyphene, dextropropoxyphene, dextromoramide, bezitramide, piritramide, methadone, dipipanone, levomethadyl acetate, difenoxin, diphenoxylate, loperamide, dezocine, pentazocine, phenazocine, buprenorphine, dihydroetorphine, etorphine, butorphanol, nalbuphine, levorphanol, levomethorphan, racemethorphan, lefetamine, menthol, meptazinol, mitragynine, tilidine, tramadol, tapentadol, eluxadoline, AP-237, or 7-hydroxymitragynine; and/or
wherein the folate treatment is vitamin B12 or folic acid; and/or
wherein the treatment for mania is lithium, quetiapine, or valproate; and/or
wherein the SMS is vilazodone or vortioxetine; and/or
wherein the vitamin B3 complex component is nicotinic acid (niacin) or nicotinamide (niacinamide); and/or
wherein the treatment for hypothyroidism is desiccated thyroid; and/or
wherein the muscle relaxant is cyclobenzaprine or tizanidine; and/or
wherein the anticonvulsant is lamotrigine, pregabalin, or gabapentin; and/or
wherein the diuretic is selected from the group consisting of (a) a thiazide-based diuretic; (b) a loop-based diuretic; (c) a potassium-sparing diuretic; (d) pamabrom; and (e) mannitol; and/or wherein the stomach medicine is selected from the group consisting of bismuth subsalicylate, calcium carbonate, and ranitidine.

18. The method of claim 17, wherein the antihistamine is administered in combination with a nonsteroidal anti-inflammatory drug (NSAID).

19. The method of claim 17, wherein the thiazide-based diuretic is indapamide, hydrochlorothiazide, chlorthalidone, chlorothiazide, metolazone, methyclothiazide, bendroflumethiazide, polythiazide, or hydroflumethiazide; and/or
wherein the loop-based diuretic is bumetanide, thacrynic acid, torsemide, or ethacrynic acid; and/or
wherein the potassium-sparing diuretic is triamterene, spironolactone, or amiloride.

20. The method of claim 17, wherein the potassium-sparing diuretic is administered with a thiazide.

21. The method of claim 20, wherein the thiazide is hydrochlorothiazide.

22. The method of claim 2 or 4, wherein the potassium-elevating agent is formulated in a pill, tablet, capsule, powder, liquid, or food conveyance.

23. The method of claim 2 or 4, wherein the potassium-elevating agent is a renin-angiotensin-aldosterone system antagonist.

24. The method of claim 23, wherein the dose of the renin-angiotensin-aldosterone system antagonist is from about 0.05 to about 600 mg/day.

25. The method of claim 23, wherein the renin-angiotensin-aldosterone system antagonist is administered to the patient in 1 to 30 dosage forms.

26. The method of claim 23, wherein the renin-angiotensin-aldosterone system antagonist is an ACE inhibitor, an angiotensin receptor antagonist, an aldosterone antagonist, or a renin inhibitor.

27. The method of claim 26, wherein the ACE inhibitor is selected from the group consisting of captopril, zofenopril, enalapril, ramipril, quinapril, perindopril, lisinopril, benazepril, imidapril, trandolapril, cilazapril, fosinopril, moexipril, spirapril, alacepril, deparil, temocapril, and teprotide; and/or
wherein the angiotensin receptor antagonist is selected from the group consisting of losartan, candesartan, valsartan, irbesartan, telmisartan, eprosartan, olmesartan, azilsartan, and firmasartan; and/or
wherein the aldosterone antagonist is selected form the group consisting of spironolactone and eplerenone; and/or
wherein the renin inhibitor is aliskiren.

28. The method of claim 23, wherein the renin-angiotensin-aldosterone system antagonist does not substantially reduce blood pressure.

29. The method of claim 23, further comprising administering a drug that increases blood pressure by a mechanism other than the renin-angiotensin-aldosterone system.

30. The method of claim 29, wherein the drug that increases blood pressure is fludrocortisone or midodrine.

31. The method of any one of claims 1-4, wherein the food conveyance is a powder, food, or beverage.

32. The method of any one of claims 1-4, wherein the food conveyance is administered to the patient in 1 to 10 dosage forms.

33. The method of claim 2 or 4, wherein the potassium-elevating agent is packaged in a kit.

34. A composition comprising an amount of potassium effective to treat a Lidocaine-Ineffective Condition, wherein the potassium is formulated in a food conveyance.

35. A kit comprising a plurality of formulations of potassium in an amount effective to treat a Lidocaine-Ineffective Condition.

36. A composition comprising potassium and an additional therapeutic agent, that are together in an amount effective to treat a Lidocaine-Ineffective Condition.

37. A kit comprising potassium and an additional therapeutic agent, wherein the potassium and additional therapeutic agent are formulated separately and in amounts effective to treat a Lidocaine-Ineffective Disorder or wherein the potassium and additional therapeutic agent are formulated together into a plurality of dosage forms in an amount effective to treat a Lidocaine-Ineffective Disorder.

38. A composition comprising a potassium-elevating agent and an additional therapeutic agent that are together in an amount effective to treat a Lidocaine-Ineffective Condition.

39. A kit comprising a potassium elevating agent and an additional therapeutic agent, wherein the potassium elevating agent and additional therapeutic agent are formulated separately and in amounts effective to treat a Lidocaine-Ineffective Disorder or wherein the potassium elevating agent and additional therapeutic agent are formulated together into a plurality of dosage forms in an amount effective to treat a Lidocaine-Ineffective Disorder.

40. The composition of claim 36 or 38 or kit of claim 37 or 39, wherein the additional therapeutic agent is a TAAR1 agonist, an inhibitor of neurotransmitter reuptake of one or more of norepinephrine, dopamine, and serotonin, an alpha-2 adrenergic receptor agonist, a monoamine oxidase inhibitor, an adenosine receptor antagonist, a barbiturate; a benzodiazepine; a hypnotic agent; an antihistamine, a pyrazolopyrimidine; a serotonin antagonist and reuptake inhibitor (SARI); a selective serotonin reuptake inhibitor (SSRI); a beta blocker; a serotonin-norepinephrine reuptake inhibitor (SNRI); a tricyclic antidepressant (TCA); a tetracyclic antidepressant; an antipsychotic; an opioid; a folate treatment; a treatment for mania; a serotonin modulator and stimulator (SMS); a vitamin B3 complex component; a treatment for hypothyroidism; a muscle relaxant; an anticonvulsant; a diuretic; or a stomach medicine.

41. The composition or kit of claim 40, wherein the TAAR1 agonist is selected from the group consisting of amphetamine, levoamphetamine, dextroamphetamine, and lisdexamfetamine;
the inhibitor of reuptake is selected from the group consisting of methylphenidate, dexmethylphenidate, atomexetine, modafinil, armodafinil, bupropion, and venlafaxine; and/or
wherein the alpha-2 adrenergic receptor agonist is selected from the group consisting of clonidine and guanfacine; and/or
wherein the monoamine oxidase inhibitor is selegiline, tranylcypromine, or phenelzine; and/or
wherein the additional therapeutic agent is selected from the group consisting of carbamazepine, pemoline, buspirone, acetaminophen, and metadoxine; and/or
wherein the adenosine receptor antagonist is selected from the group consisting of caffeine, theophylline, and theobromine; and/or
wherein the additional therapeutic agent is selected from the group consisting of acontium napellus, chocolate, cinchona officinalis, coffee, gnaphalium polycephalum, guarana, guayusa, leduum palustre, magnesia phosphorica, rhus toxicodendron, tea, viscum album, Hypericum, yaupon, and khat; and/or
wherein the additional therapeutic agent is a nonsteroidal anti-inflammatory drug (NSAID); and/or
wherein the NSAID is aspirin, diclofenac, diflunisal, indomethacin, sulindac, etodolac, mefenamic acid, meclofenamate, flufenamic acid, tolmetin, ketorolac, diclofenac, ibuprofen, naproxen, fenoprofen, ketoprofen, flurbiprofen, oxaprozin, piroxicam, meloxicam, nabumetone, celecoxib, valdecoxib, parecoxib, etoricoxib, or lumaricoxib; and/or
wherein the barbiturate is secobarbital, pentobarbital, phenobarbital, amobarbital, or butabarbital; and/or
wherein the benzodiazepine is alprazolam, diazepam, lorazepam, temazepam, clonazepam, oxazepam, quazepam, flurazepam, adinazolam, estazolam, flubromazolam, nitrazolam, pyrazolam, triazolam, or zapizolam; and/or
wherein the hypnotic agent is chloral hydrate, eszopiclone, tasimelteon, zolpidem, ramelteon, SAR, melatonin, agomelatine, tasimelteon, TIK-301, or suvorexant; and/or
wherein the antihistamine is acrivastine, azelastine, acrivastine, cetirizine, diphenhydramine, bilastine, bromodiphenhydramine, brompheniramine, buclizine, carbinoxamine, chlorodiphenhydramine, chlorphenamine, chlorpromazine, clemastine, cyclizine, cyproheptadine, dexbrompheniramine, dexchlorpheniramine, dimenhydrinate, dimetindene, doxylamine, ebastine, embramine, fexofenadine, loratidine, hydroxyzine, meclizine, mirtazapine, olopatadine, orphenadrine, phenindamine, pheniramine, phenyltoloxamine, promethazine, rupatadine, tripelennamine, or triprolidine; and/or
wherein the pyrazolopyrimidine is zaleplon, indiplon, ocinaplon, divaplon, or lorediplon; and/or
wherein the SARI is trazodone, nefazodone, mepiprazole, lubazodone, loriprazole, or etoperidone; and/or
wherein the SSRI is sertraline, escitalopram, fluoxetine, citalopram, or paroxetine; and/or wherein the beta blocker is propranolol or atenolol; and/or
wherein the SNRI is duloxetine, venlafaxine, desvenlafaxine, atomozetine, milnacipran, or levomilnacipran; and/or
wherein the TCA is nortriptyline, imipramine, amoxapine, desipramine, dibenzocycloheptadiene, trimipramine, doxepin, amitriptyline/chlordiazepoxide, clomipramine, amitriptyline/perphenazine, or protriptyline; and/or
wherein the tetracyclic antidepressant is mirtazapine, maprotiline, or a piperazino-azepine; and/or
wherein the antipsychotic is aripiprazole, olanzapine, risperidone, paliperidone, or brexipiprazole; and/or
wherein the opioid is codeine, morphine, thebaine, oripavine, diacetylmorphine, nicomorphine, dipropanoylmorphine, diacetyldihydromorphine, acetylpropionylmorphine, desomorphine, methyldesorphine, dibenzoylmorphine, dihydrocodeine, ethylmorphine, heterocodeine, buprenorphine, etorphine, hydrocodone, hydromorphone, oxycodone, oxymorphone, fentanyl, alphamethylfentanyl, alfentanil, sufentanil, remifentanil, carfentanyl, ohmefentanyl, pethidine, ketobemidone, mppp, allylprodine, prodine, pepap, promedol, propoxyphene, dextropropoxyphene, dextromoramide, bezitramide, piritramide, methadone, dipipanone, levomethadyl acetate, difenoxin, diphenoxylate, loperamide, dezocine, pentazocine, phenazocine, buprenorphine, dihydroetorphine, etorphine, butorphanol, nalbuphine, levorphanol, levomethorphan, racemethorphan, lefetamine, menthol, meptazinol, mitragynine, tilidine, tramadol, tapentadol, eluxadoline, AP-237, or 7-hydroxymitragynine; and/or
wherein the folate treatment is vitamin B12 or folic acid; and/or
wherein the treatment for mania is lithium, quetiapine, or valproate; and/or
wherein the SMS is vilazodone or vortioxetine; and/or
wherein the vitamin B3 complex component is nicotinic acid (niacin) or nicotinamide (niacinamide); and/or
wherein the treatment for hypothyroidism is desiccated thyroid; and/or
wherein the muscle relaxant is cyclobenzaprine or tizanidine; and/or
wherein the anticonvulsant is lamotrigine, pregabalin, or gabapentin; and/or
wherein the diuretic is selected from the group consisting of (a) a thiazide-based diuretic; (b) a loop-based diuretic; (c) a potassium-sparing diuretic; (d) pamabrom; and (e) mannitol; and/or wherein the stomach medicine is selected from the group consisting of bismuth subsalicylate, calcium carbonate, and ranitidine.

42. The composition or kit of claim 41, wherein the antihistamine is administered in combination with a nonsteroidal anti-inflammatory drug (NSAID).

43. The composition or kit of claim 41, wherein the thiazide-based diuretic is indapamide, hydrochlorothiazide, chlorthalidone, chlorothiazide, metolazone, methyclothiazide, bendroflumethiazide, polythiazide, or hydroflumethiazide; and/or
wherein the loop-based diuretic is bumetanide, thacrynic acid, torsemide, or ethacrynic acid; and/or
wherein the potassium-sparing diuretic is triamterene; spironolactone, or amiloride.

44. The composition or kit of claim 43, wherein the potassium-sparing diuretic is administered with a thiazide.

45. The composition or kit of claim 44, wherein the thiazide is hydrochlorothiazide.

46. The composition of claim 38 or kit of claim 39, wherein the potassium-elevating agent is a renin-angiotensin-aldosterone system antagonist.

47. The composition or kit of claim 46, wherein the dose of the renin-angiotensin-aldosterone system antagonist is from about 0.05 to about 600 mg/day.

48. The kit of claim 39, wherein the renin-angiotensin-aldosterone system antagonist is present in 2 to 30 dosage forms.

49. The composition of claim 38 or kit of claim 39, wherein the renin-angiotensin-aldosterone system antagonist is an ACE inhibitor, an angiotensin receptor antagonist, an aldosterone antagonist, or a renin inhibitor.

50. The composition or kit of claim 49, wherein the ACE inhibitor is selected from the group consisting of captopril, zofenopril, enalapril, ramipril, quinapril, perindopril, lisinopril, benazepril, imidapril, trandolapril, cilazapril, fosinopril, moexipril, spirapril, alacepril, deparil, temocapril, and teprotide; and/or
wherein the angiotensin receptor antagonist is selected from the group consisting of losartan, candesartan, valsartan, irbesartan, telmisartan, eprosartan, olmesartan, azilsartan, and firmasartan; and/or
wherein the aldosterone antagonist is selected form the group consisting of spironolactone and eplerenone; and/or
wherein the renin inhibitor is aliskiren.

51. The composition or kit of claim 46, wherein the renin-angiotensin-aldosterone system antagonist does not substantially reduce blood pressure.

52. The composition or kit of claim 46, further comprising administering a drug that increases blood pressure by a mechanism other than the renin-angiotensin-aldosterone system.

53. The composition or kit of claim 52, wherein the drug that increases blood pressure is fludrocortisone or midodrine.

54. The composition of claim 36 or kit of claim 37, wherein the potassium is formulated for extended release.

55. The composition of claim 36 or 38, formulated as a food conveyance.

56. The kit of claim 37 or 39, wherein the dosages are formulated as food conveyances.

57. The composition of claim 36 or kit of claim 37, wherein the composition comprises 90 mg to 5,000 mg of elemental potassium.

58. The kit of claim 37 or 39, comprising between 1 and 30 dosages of potassium or potassium elevating agent.
